(19) United States
(12) Patent Application Publication
(10) Pub. No.: US 2026/0218271 A1
(43) Pub. Date: Jul. 30, 2026

(54) MODIFIED IMMUNO-MOLECULAR ASSAY FOR FASTER BIOMARKER DETECTION

(71) Applicant: BIOMÉRIEUX, Marcy L'Etoile (FR)

(72) Inventors: Frédéric BEDIN, Lyon (FR); Vincent BENOIT, Lyon (FR); Agnès RUBENS, Montceaux (FR)

(73) Assignee: BIOMÉRIEUX, Marcy L'Etoile (FR)

(21) Appl. No.: 19/146,123

(22) PCT Filed: Jan. 26, 2024
(86) PCT No.: PCT/EP2024/051975

§ 371 Date: Jul. 7, 2025

(30) Foreign Application Priority Data
Jan. 27, 2023 (EP) 23305109.3

(51) Int. Cl. C12Q 1/6804

(52) U.S. Cl. CPC C12Q 1/6804

(57) ABSTRACT

The present invention relates to an improved immuno-molecular assay, for example a Proximity Extension Assay protocol or a Proximity Ligation Assay (PLA) protocol, which is more compatible with the assay durations that are expected in the context of point-of-care diagnostics. In particular, the time needed for performing the Proximity Extension Assay protocol of the invention is reduced by more than 13-folds as compared to classical Proximity Extension Assay protocols, without significantly affecting the performance of the assay for protein detection. Thus, the method of the invention provides a more rapid and simpler procedure to screen and diagnose diseases in a non-invasive way. The use of such a method in an integrated system is an exciting and expected improvement in clinical diagnostics for the detection of biomarkers in human samples.

### BRIEF SUMMARY

The present invention relates to an improved immuno-molecular assay, for example a Proximity Extension Assay protocol or a Proximity Ligation Assay (PLA) protocol, which is more compatible with the assay durations that are expected in the context of point-of-care diagnostics. In particular, the time needed for performing the protocol of the invention is reduced by more than 13-folds as compared to classical Proximity Extension Assay protocols, without significantly affecting the performance of the assay for protein detection. Thus, the method of the invention provides a more rapid and simpler procedure to screen and diagnose diseases in a non-invasive way. The use of such a method in an integrated system is an exciting and expected improvement in clinical diagnostics for the detection of biomarkers in human samples.

### DESCRIPTION OF THE PRIOR ART

The discovery and application of blood biomarkers play a central role in clinical medicine. The ability to identify disease-related physiological processes from a blood sample contributes to effective clinical diagnosis. While analyses at the transcript level often require sampling or biopsy, biomarker analysis can allow for sufficiently sensitive diagnosis, prognosis or stratification of patients by simple blood sampling.

In this regard, the sandwich ELISA technique is very popular in clinical practice [1]. These assays are most often performed using an immobilized capture antibody, while the captured target protein is revealed by binding to second antibodies conjugated to enzymes, providing a detectable signal by enzymatic conversion of substrate molecules. One major drawback of ELISA is that it is not well suited for multiplex measurements since cross-binding of antibodies can interfere with the signal reading. This problem increases exponentially with the degree of multiplexing.

Immuno-PCR (i-PCR), first described in 1992 [2], involves the amplification of linear DNA bound to a specific monoclonal antibody, and a second specific antibody immobilized on microtiter-plate wells or magnetic beads. While the first versions of i-PCR required time-consuming and labour-intensive post-PCR analysis, the use of qPCR has not only simplified i-PCR protocols, but also increased the dynamic range of the assay [3]. Today, i-PCR is often considered to be a robust method that offers the specificity and sensitivity required for many clinical applications, including the detection of biomarkers of diagnostic interest [4]. The main drawback of i-PCR is its non-homogeneous nature, which requires numerous wash steps to ensure minimal background.

Proximity assays, developed more recently, largely address this problem. The Proximity Ligation Assay (hereafter referred to as “PLA”) was first documented in 2002 [5]. PLA uses probes that are usually obtained by covalent attachment of two separate oligonucleotides to two antibodies specific for the biomarker to be detected. Briefly, the workflow consists in the binding of the antibodies to the biomarker via their epitopes, followed by a ligation step that generates amplifiable DNA templates, and finally signal generation that can be based on different readout formats. PLA can be performed in solution as a homogeneous assay, which has the advantage of minimizing operator intervention, negating the need for washes and thus facilitating shorter assay times. Like i-PCR, PLA assays can also be configured in a solid-phase format using an immobilized capture antibody, with two proximity probes binding to the captured target molecules [6], an approach that may be more suitable for detecting proteins directly from biofluids such as blood or feces. Multiplexed PLAs have already been developed [7]. The main disadvantage of PLA is related to the use of DNA ligase, an enzyme with performance dependent on the incubation temperature and very sensitive to inhibitors present in the biological samples to be analysed.

Proximity Extension Assay is an alternative to PLA and was developed because proximity probes joined by a DNA ligase suffer from recovery loss in complex biological fluids [8]. The main difference between the two methods is that in Proximity Extension Assay the ligation event is replaced by a DNA polymerisation step. The analyte is detected by the binding of at least two specific probes, which, when brought in close proximity one to the other (after binding to the analyte), allow a nucleotide extension product to be generated and possibly quantified. Proximity Extension Assay is readily amenable to multiplexing [9] and features the same advantages as PLA, including very low sample consumption, high sensitivity (down to 1-10 μg/mL) and specificity, and detection in a homogeneous reaction. For maximal sensitivity, Proximity Extension Assay can use DNA polymerases with 3′>5′ exonuclease activity as they reduce background noise by degrading non-proximal DNA strands. Furthermore, judicious choice of DNA polymerase can mediate minimisation of background noise and so improves the sensitivity of the assay [9].

Because it operates in a liquid homogeneous phase, at a single temperature and on a very small sample volume, Proximity Extension Assay has the required characteristics to be used in decentralized diagnostic systems, near the patient. Proximity Extension Assay combines the specificity of an antibody-based immunoassay with the sensitivity of polymerase chain reaction (PCR), and real-time quantitative PCR (qPCR) readout. The result is a flexible, highly specific and sensitive method that can be used to simultaneously quantify multiple biomarkers in a single sample.

Yet, the PLA and Proximity Extension Assay described in the literature are limited by the duration of the assays, which is too long to be used in routine in point-of-care laboratories (the current assays last for more than 90 min, qPCR excluded, due to multiple dilution and incubation steps in different buffers). This is because PLA/Proximity Extension Assay has been developed as a tool to characterize biomarkers in proteomic applications, for example as multiplex tests (up to 98-plex), in research laboratories. So far, little work has been done to adapt the PLA/Proximity Extension Assay protocols for diagnostic applications with demanding requirements in terms of sensitivity, specificity and minimization of assay time.

However, as the clinical management of patients with severe infections or sepsis is always critical and time-dependent, the availability of fully automated immunoassays with high throughput, short time-to-result, low sample volume, and reasonable cost, is essential. The present invention solves this need.

As a matter of fact, the present inventors herein propose a simpler and shortened PLA/Proximity Extension Assay protocol, which is more compatible with the assay durations that are expected in the context of point-of-care diagnostics. In particular, they were able to reduce the time needed for Proximity Extension Assay by more than 13-folds without significantly affecting the performance of the assay for protein detection. Importantly, the sensitivity of this improved assay was determined to be of about 50-100 μg/mL of protein in plasma specimen.

The method of the invention provides a more rapid and simpler procedure to screen and diagnose diseases in a non-invasive way. The use of such a method in an integrated system is an exciting and expected improvement in clinical diagnostics for the detection of biomarkers in human samples.

### SUMMARY

An object of the disclosure relates to an immuno-molecular assay for detecting a protein analyte or a fragment thereof in a biological sample, said assay comprising the steps of A) contacting said biological sample with at least one molecule comprising a nucleic acid domain and a binding domain being able to directly or indirectly bind to said protein analyte, B) incubating the resulting sample from 1 minute to 30 minutes under intermittent shaking for allowing the at least one molecule to bind to the protein analyte, and C) detecting the binding by a molecular detection method. By intermittent shaking, it is meant a shaking with alternating periods of agitation and stagnation (static period). In one embodiment, the sample is incubated in step B) from about 3 minutes to about 25 minutes, preferably from about 5 minutes to about 20 minutes, and more preferably from about 5 minutes to about 15 minutes under intermittent shaking.

In a preferred embodiment, the intermittent shaking of step B) comprises alternating periods of shaking lasting from 5 seconds to 30 seconds and static periods lasting from 2 to 3 minutes. In step A), the at least one molecule is preferably at least one set of a first and a second proximity probes, each proximity probe comprising an analyte-binding domain and a nucleic acid domain, said analyte-binding domains binding directly to the same protein analyte, and said nucleic acid domains being able to hybridize.

In one particular embodiment, the method of the invention comprises the steps of: I). Contacting said biological sample with at least one set of a first and a second proximity probes, each proximity probe comprising an analyte-binding domain and a nucleic acid domain, said analyte-binding domains binding to the same analyte, and said nucleic acid domains being able to hybridize, II). Incubating the resulting sample from 3 to 30 minutes under intermittent shaking, for allowing the proximity probes to bind to the protein analyte and the nucleic acid domains to hybridize with each other to form a duplex structure, III). Diluting the resulting mixture sample with a buffer and incubating further the diluted sample, IV). Adding a DNA polymerase to the sample, and allowing the extension of the 3′ end of at least one nucleic acid domain of said duplex to generate an extension product, V). Heat inactivating said DNA polymerase, VI). Detecting the extension product.

In this method, the DNA polymerase added in step IV) is preferably chosen in the group consisting of: T4 DNA polymerase, T7 DNA polymerase, Extaq polymerase combined with another enzyme having 3′>5′ exonuclease activity, Phi29 (@29) DNA polymerase, DNA polymerase I, Klenow fragment of DNA polymerase I, Pyrococcus furiosus (Pfu) DNA polymerase and Pyrococcus woesei (Pwo) DNA polymerase.

Moreover, the inactivation step V) is preferably achieved at a temperature comprised between 65° C.-80° C., more preferably from 10 seconds to 3 minutes, even more preferably during 30 seconds. Also, in step II) the sample is preferably incubated from about 3 min to 5 min under intermittent shaking.

Finally, the detecting step VI) is preferably performed by PCR, more preferably by qPCR. This method comprises preferably a multiplex analysis using several sets of at least first and second proximity probes, wherein each set is specific of a particular protein analyte and produces a unique extension product.

In another particular embodiment, the at least one molecule in step A) can be at least one set of two proximity probes, each proximity probe comprising an analyte-binding domain and a nucleic acid domain, said analyte-binding domains binding directly to the same protein analyte, and said nucleic domains being able to be ligated together.

Alternatively, the at least one molecule in step A) can be a first set of at least two proximity probes each comprising an analyte-binding domain and a second set of at least two proximity probes each comprising a binding domain able to bind to at least one probe of the first set of proximity probes and a nucleic acid domain, said nucleic domains being able to be ligated together. In this case, the method of the invention preferably comprises the steps of: i) Contacting said biological sample with at least one set of two proximity probes, each proximity probe comprising an analyte-binding domain and a nucleic acid domain, said analyte-binding domains binding directly to the same protein analyte, and said nucleic domains being able to be ligated together, or contacting said biological sample with a first set of at least two proximity probes each comprising an analyte-binding domain and with a second set of at least two proximity probes each comprising a binding domain able to bind to at least one probe of the first set of proximity probes and a nucleic acid domain, said nucleic domains being able to be ligated together, j) Incubating the resulting sample from 1 to 30 minutes under intermittent shaking for allowing the at least one molecule to bind to the protein analyte, k) Ligating the nucleic acid domains of the proximity probes by a ligase enzyme, and I) Detecting the ligated nucleic acids. In a preferred embodiment, in this method, the analyte binding domain of said proximity probes is an antibody, or a binding fragment thereof or a derivative thereof.

For all these methods, the biological sample is preferably serum or plasma.

### DETAILED DESCRIPTION OF THE INVENTION

The main objective of the work presented here was to optimize immuno-molecular assays, in particular Proximity Extension Assays and Proximity Ligation Assays (PLA) for the detection of markers of medical interest in human samples. More precisely, the aim was to establish the proof of principle of a PLA/Proximity Extension Assay protocol that would have a duration more compatible with the assay times that are required in the context of clinical diagnostics (typically less than 30 min). An optimized Proximity Extension Assay protocol was thus designed, which is simpler and faster than the Proximity Extension Assay protocols of the prior art.

The present invention relates to a short immuno-molecular assay, such as Proximity Extension Assay or PLA assay, that enables quantification of a biomarker or an analyte over a large range of concentrations and with low limit of detection (0.1 ng/ml) in a complex sample, e.g. human plasma.

In one preferred embodiment, the analyte detected by the assay in the biological sample is the procalcitonin (PCT).

### Main Steps of Classical Protocols of Proximity Extension Assay

Proximity Extension Assays were first described by Lundberg et al. in 2011 [8], for the detection of protein biomarkers in liquid samples. For each protein biomarker, a pair of antibodies conjugated to unique oligonucleotides (hereafter referred to as “Proximity Extension Assay probes”) binds to their respective epitope. As a result, the probes approach each other by proximity and hybridize through the oligonucleotide 3′-ends. The subsequent addition of DNA polymerase leads to an extension step from this template, creating a DNA duplex that can then be detected and quantified by PCR, preferably by real-time quantitative PCR (qPCR). The signal at the end of the assay is quantitatively proportional to the initial concentration of the target protein.

As used herein, the term “Proximity Extension Assay” therefore refers to a specific type of assay, which utilises the following steps:
a) Contacting an analyte with multiple (i.e. two or more, generally two or three) probes, each comprising a nucleic acid domain (or moiety) linked to the analyte-binding domain (or moiety) of the probe,
b) Allowing interaction between the nucleic acid moieties and/or a further functional moiety which are carried by the other probe(s), when the probes have bound to the analyte, so as to form a duplex DNA molecule,
c) Detecting the interaction of step b) by performing the three following independent steps:
   c1) extending one of the bound nucleic acid domain from the duplex to generate a detectable extension product, by adding at least one DNA polymerase enzyme, comprising advantageously a 3′>5′ exonuclease activity, and dNTPs in the sample,
   c2) inactivating the polymerase and other enzymes present in the sample,
   c3) detecting the extension product generated by the extension step c1).

As used herein, the term “extension product” designates the oligonucleotide duplex molecule resulting from full or partial extension of the nucleic acid domain of one proximity probe, the extended part being complementary to the nucleic acid domain of the other proximity probe, which act as the template. Accordingly, the maximum size of the extension product will depend on the size of the nucleic acid domains of the proximity probes which act as the template for the extension product.

Once produced, the extension product produced by the above-described Proximity Extension Assay may, in the broadest sense, be detected using any convenient protocol. The particular detection protocol may vary depending on the sensitivity desired and the application in which the method is being practiced. In certain embodiments, the extension product may be directly detected without any amplification, while in other embodiments the detection protocol may include an amplification step, in which the copy number of the extension product is increased, e.g., to enhance sensitivity of the particular assay.

Where detection without amplification is practicable, the extension product may be detected in a number of different ways. For example, one or more of the nucleic acid domains of the proximity probes may be directly labelled, e.g., fluorescently, or otherwise spectrophotometrically, or radioisotopically labelled or with any signal-giving label, such that the extension product is directly labelled. In these embodiments, the directly labelled extension product may be size separated from the remainder of the reaction mixture. Alternatively, conformationally selective probes, e.g., molecular beacons may be employed to detect to the presence of the extension products, where these probes are directed to a sequence that spans the extension product and therefore only exists in its entirety in the extension products.

In a preferred embodiment, however, the detection protocol includes an amplification reaction in which the copy number of the extension product nucleic acid (or part thereof) is increased. The amplification may be linear or exponential, as desired, where representative amplification protocols of interest include, but are not limited to: polymerase chain reaction (PCR); isothermal amplification, Rolling circle amplification, etc. All these protocols are well-known in the art.

In a particular embodiment, the final step of the Proximity Extension Assay consists in amplifying the extension product by PCR or qPCR, so as ensure that it can be detected (its existence meaning that the analyte was present in the sample).

The term “amplifying” is used generally herein to include any means of increasing the number of copies of the extension product or part thereof. Any amplification means known in the art may be utilised in the methods of the invention, e.g. PCR, LCR, RCA, MDA etc. Depending on the abundance of the target analyte in the sample, it may be necessary to amplify the extension product, or part thereof, such that the concentration of the extension product has doubled, i.e., 2 times the number of copies present before amplification. Alternatively, it may be preferable to increase the number of copies by multiple orders of magnitude. In some embodiments amplification results in the sample comprising at least 2, 3, 4, 5, 6, 7, 8, 9, 10, 15, 20, 50 or 75 times the original amount of extension product or part thereof. In further preferred embodiments it may be preferable to amplify the extension product such that the sample comprises at least 102, 103, 104, 105, 106, 107, 108, 109, 1010 etc. times the original amount of extension product or part thereof.

The extension product produced by the Proximity Extension Assay effectively comprises two parts: a first “old” part (the existing part) containing the nucleotide sequence that made up the nucleic acid domain of the proximity probe and a second “new” part (the extended part) containing the nucleotide sequence generated by the templated extension reaction. It is the detection of the second “new” or “extended” part that allows the detection of the of the target analyte, because, if there is no analyte, there will be no extension and hence no “new” or “extended” part. Thus, in a preferred aspect of the invention, the step of amplifying the extension product comprises amplifying a portion of the extended part of the extension product, that was not present in the sample before the extension reaction occurred. The skilled person will be able to determine the size of the portion of the extended part that is sufficient to be amplified so as to distinguish it from other sequences present in the sample. Preferably, the portion may comprise at least 8 nucleotides, preferably at least 9, 10, 11, 12, 13, 14, 15, 16, 17, 18, 19, 20 nucleotides of the extended part of the extension product.

### Main Steps of Classical Protocols of PLA

PLA assays use probes that are usually obtained by covalent attachment of two separate oligonucleotides to two antibodies specific for the protein to be detected. Briefly, the workflow consists in the binding of the antibodies to the protein via their epitopes, followed by a ligation step that generates amplifiable DNA templates, and finally signal generation that can be based on different readout formats.

Direct PLA assays involve only one set of proximity probes (the probes containing both a protein binding domain and a nucleic acid domain). Indirect PLA assays involve two sets of proximity probes (one set binding to the target protein, one set containing a nucleic acid domain on a one hand, and being able to bind the probes of the first set on the other hand).

As used herein, the term “PLA” thus refers to a specific type of assay, which utilises the following steps:
i) Contacting an analyte with one or two sets of probes, typically:
with a set of two or more (generally two or three) proximity probes comprising an analyte-binding domain (or moiety) and a nucleic acid domain (or moiety), said analyte-binding domains binding directly to the same protein analyte, and said nucleic acid domains being able to be ligated together, or
with two sets of proximity probes, one first set of proximity probes containing two or more (generally two or three) proximity probes comprising an analyte-binding domain, and a second set of two or more (generally two or three) proximity probes, each proximity probe of said second set comprising a binding domain that binds to at least one probe of the first set, and a nucleic acid domain (or moiety), the nucleic acid domains of said at least two proximity probes of the second set being able to be ligated together,
j) Allowing interaction between the nucleic acid moieties of the proximity probes,
k) Ligating the nucleic acid domains of the proximity probes by a ligase enzyme, so as to create a closed, circular DNA template,
l) Detecting the ligated nucleic acids.

The ligated product produced by the above-described PLA may, in the broadest sense, be detected using any convenient protocol. The particular detection protocol may vary depending on the sensitivity desired and the application in which the method is being practiced. In certain embodiments, the ligated nucleic acids may be directly detected without any amplification, while in other embodiments the detection protocol may include an amplification step, in which the copy number of the ligated nucleic acids is increased, e.g., to enhance sensitivity of the particular assay. Where detection without amplification is practicable, the ligated nucleic acids may be detected in a number of different ways. For example, one or more of the nucleic acid domains of the proximity probes may be directly labelled, e.g., fluorescently, or otherwise spectrophotometrically, or radioisotopically labelled or with any signal-giving label, such that the ligation product is directly labelled. In these embodiments, the directly labelled ligation product may be size separated from the remainder of the reaction mixture, including unligated directly labelled ligation oligonucleotides (i.e., marker oligonucleotides or cassette oligonucleotides), in order to detect the ligated nucleic acids. Alternatively, conformationally selective probes, e.g., molecular beacons may be employed to detect to the presence of the ligated nucleic acids, where these probes are directed to a sequence that spans the ligated nucleic acids and therefore only exists in its entirety in the ligated nucleic acids.

In a preferred embodiment, step I) involves an amplification step, where the copy number of the ligated nucleic acids is increased, e.g., in order to enhance sensitivity of the assay. The amplification may be linear or exponential, as desired. This amplification step is advantageously performed by rolling circle-amplification, as described in WO 2012/160083. Other amplification methods, e.g., classical PCR, NASBA (Nucleic Acid Sequence-Based Amplification), LCR (Ligase chain reaction), SMAP (SMart Amplification Process), or HDA (Helicase-Dependent Amplification) may also be used. Afterwards, the circle is preferred detected by means of detection oligonucleotides which hybridise to detection tags that were present in the nucleic acid domains of the proximity probes, or to their complements in the amplification product of the circle, for example with fluorescently labelled detection oligonucleotides.

The ligase enzyme that is used in PLA assays is selected on the basis that it cannot ligate mismatched sequences. As is known in the art, ligases catalyze the formation of a phosphodiester bond between juxtaposed 3′-hydroxyl and 5′-phosphate termini of two immediately adjacent nucleic acids when they are annealed or hybridized to a third nucleic acid sequence to which they are complementary (i.e. a template). Any convenient ligase may be employed, where representative ligases of interest include, but are not limited to, temperature sensitive and thermostable ligases. Temperature sensitive ligases include, but are not limited to, bacteriophage T4 DNA ligase, bacteriophage T7 ligase, and E. coli ligase. Thermostable ligases include, but are not limited to, Taq ligase, Tth ligase, and Pfu ligase. Thermostable ligase may be obtained from thermophilic or hyperthermophilic organisms, including but not limited to, prokaryotic, eukaryotic, or archae organisms. Certain RNA ligases may also be employed.

Tools that can be used in the assays PLA/Proximity Extension Assay

### Probes

The probes used in the PLA/Proximity Extension Assay methods of the invention are herein called “proximity probes” or “PLA/Proximity Extension Assay probes”. They have been well characterized in the prior art. They are generally used in pairs, and individually consist of an analyte-binding domain having a strong specificity for the target analyte, and a functional domain, e.g. a nucleic acid domain, coupled thereto. This is the case for Proximity Extension Assays and for direct PLA assays. The probes used in Proximity Extension Assay and in direct PLA assays have a similar structure. We will call them “Proximity Extension Assay/direct PLA probes” in the following.

For indirect PLA assays, two sets of proximity probes should be used: the probes of a first set comprise only an analyte-binding domain, and the probes of the second set comprise a nucleic acid domain (or moiety) and a binding domain that binds to the probes of the first set. These probes will be called “indirect PLA probes” in the following.

The respective analyte-binding domains of each proximity probe pair may have specificity for different binding sites on the analyte, or may have identical specificities, for example in the event that the target analyte exists as a multimer.

Modifications of the structure of proximity probes have been described, for example in WO 03/044231, where multivalent proximity probes are described. Such multivalent proximity probes may comprise as many as 100 analyte-binding domains conjugated to at least one, and preferably more than one, nucleic acid(s).

The sensitivity of PLA/Proximity Extension Assay is limited by two main factors: (i) the affinity of the analyte-binding domains for the target analyte and (ii) the non-specific background signal arising from the random proximity of non-bound probes, particularly probe pairs. To reduce these limitations, it is recommended using probes having binding domains having a high affinity for the analyte so that very low concentrations of proximity probes can be used.

With the methods of the invention, a reaction mixture may readily be screened (or assessed or assayed etc.) for the presence of several target analytes. These methods are indeed suitable for the detection of a single target analyte as well as multiplex analyses, in which two or more different target analytes are assayed in the sample. In these latter multiplex situations, the number of different sets of probes that may be employed typically ranges from about 2 to about 20 or higher, e.g., as up to 50.

In the context of the invention, the Proximity Extension Assay/direct PLA probes comprise a nucleic acid domain that is coupled to an analyte-binding domain by any means in the art (by direct or indirect means, via covalent binding or via a linking group such as streptavidin-biotin coupling).

### Analyte-Binding Domain

The analyte binding domain may be any binding partner for the target analyte, and it may be also a direct or an indirect binding partner for the analyte, provided that it is sufficiently specific and has high affinity for the analyte. By “high binding affinity” it is meant a binding affinity of at least about 104 M, usually at least about 106 M or higher, e.g., 109 M or higher. The analyte binding domain may be any of a variety of different types of molecules, so long as it exhibits the requisite binding affinity for the target protein when present as part of the proximity probe.

The analyte binding domain may be a small or a large molecule (ranging in size from about 50 to about 10,000 daltons, or of greater molecular weight). Of particular interest as large molecule analyte binding domains are antibodies, as well as binding fragments and derivatives or mimetics thereof. Where antibodies are the analyte binding domain, they may be derived from polyclonal compositions, such that a heterogeneous population of antibodies differing by specificity are each “tagged” with the same tag nucleic acid (nucleic acid domain) or monoclonal compositions, in which a homogeneous population of identical antibodies that have the same specificity for the target analyte are each tagged with the same tag nucleic acid. As such, the analyte binding domain may be either a monoclonal or polyclonal antibody. In yet other embodiments, the affinity ligand is an antibody binding fragment or derivative or mimetic thereof, where these fragments, derivatives and mimetics have the requisite binding affinity for the target analyte. For example, antibody fragments, such as Fv, F (ab) 2 and Fab may be prepared by cleavage of the intact protein, e.g. by protease or chemical cleavage. Also of interest are recombinantly or synthetically produced antibody fragments or derivatives, such as single chain antibodies or scFvs, or other antibody derivatives such as chimeric antibodies or CDR-grafted antibodies, where such recombinantly or synthetically produced antibody fragments retain the binding characteristics of the above antibodies. Such antibody fragments or derivatives generally include at least the VH and VL domains of the subject antibodies, so as to retain the binding characteristics of the subject antibodies.

Such antibody fragments, derivatives or mimetics of the subject invention may be readily prepared using any convenient methodology, such as the methodology disclosed in U.S. Pat. Nos. 5,851,829 and 5,965,371.

The above-described antibodies, fragments, derivatives and mimetics thereof may be obtained from commercial sources and/or prepared using any convenient technology, where methods of producing polyclonal antibodies, monoclonal antibodies, fragments, derivatives and mimetics thereof, including recombinant derivatives thereof, are known to those of the skill in the art.

Also suitable for use as binding domains are polynucleic acid aptamers. Polynucleic acid aptamers may be RNA oligonucleotides which may act to selectively bind proteins, much in the same manner as a receptor or antibody ([10]). In certain embodiments where the analyte binding domain is a nucleic acid, e.g., an aptamer, the target analyte is not a nucleic acid.

In addition to antibody-based peptide/polypeptide or protein-based binding domains, the analyte binding domain may also be a lectin, a soluble cell-surface receptor or derivative thereof, an affibody or any combinatorially derived protein or peptide from phage display or ribosome display or any type of combinatorial peptide or protein library.

The analyte-binding domain may bind to the analyte directly or indirectly. In the case of indirect binding, the target analyte may first be bound by a specific binding partner (or affinity ligand), and the analyte-binding domain of the proximity probe may bind to the specific binding partner. This enables the design of proximity probes as universal reagents. For example, the analyte-specific binding partner may be an antibody, and a universal proximity probe set may be used to detect different analytes by binding to the Fc regions of the various different analyte-specific antibodies.

### Nucleic Acid Domain (Functional Domain)

When a proximity probe pair come into close proximity with each other, which will primarily occur when both are bound to their respective sites on the same analyte molecule, the nucleic acid domains of the probes interact because they contain complementary regions at the 3′ ends and hence may hybridise to form a duplex, or may be ligated together by means of a ligase.

In the context of the invention, the nucleic acid domains may be a single stranded nucleic acid molecule (e.g. an oligonucleotide), a partially double stranded and partially single stranded molecule, or a double stranded molecule that includes a region that is double stranded and a region where the two nucleic acid strands are not complementary and therefore single stranded. As such, in certain embodiments, the nucleic acid domain is made up of a single stranded nucleic acid. In other embodiments, the nucleic acid domain may be made up of two partially complementary nucleic acid strands, where the two strands include a hybridized region and non-hybridized region.

For Proximity Extension Assay according to the invention, the nucleic acid domains of the proximity probes must be capable of interaction by hybridisation. The term “hybridisation” or “hybridises” as used herein refers to the formation of a duplex between nucleotide sequences which are sufficiently complementary to form duplexes via Watson-Crick base pairing. Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind to a section of a second sequence in an anti-parallel sense wherein the 3′-end of each sequence binds to the 5′-end of the other sequence and each A, T (U), G and C of one sequence is then aligned with a T (U), A, C and G, respectively, of the other sequence. RNA sequences can also include complementary A=U or U=A base pairs. Thus, two sequences need not have perfect homology to be “complementary” under the invention. Usually two sequences are sufficiently complementary when at least about 85% (preferably at least about 90%, and most preferably at least about 95%) of the nucleotides share base pair organization over a defined length of the molecule or the domains that are determined to be complementary. The nucleic acid domains of the proximity probes thus contain a region of complementarity for the nucleic acid domain of the other proximity probe.

Still for Proximity Extension Assay, the nucleic acid domains of the proximity probes are therefore made up of ribonucleotides and/or deoxyribonucleotides as well as synthetic nucleotide residues that are capable of participating in Watson-Crick type or analogous base pair interactions, i.e. “hybridisation” or the formation of a “duplex”. Thus, the nucleic acid domain may be DNA or RNA or any modification thereof e.g. PNA or other derivatives containing non-nucleotide backbones. This interaction may be direct, e.g. the nucleic acid domains comprise regions of complementarity to each other, preferably at their 3′ ends (although the region of complementarity may be internal to one nucleic acid domain), or indirect, e.g. the nucleic acid domains of said first and second proximity probes may each hybridise with a region of a so-called “splint” oligonucleotide. The sequence of the nucleic acid domain of the proximity probes is not critical as long as the two domains may hybridise to each other. However, the sequences should be chosen to avoid the occurrence of hybridization events other than between the nucleic acid domains of the proximity probes or with that of the splint oligonucleotide. Once the sequence is selected or identified, the nucleic acid domains may be synthesized using any convenient method.

For the PLA according to the invention, the nucleic acid domains of the proximity probes must be capable of being ligated by an enzyme (e.g. ligase). To do so, the 3′-end of a first nucleic acid domain has an hydroxyl group (3′-OH) and the 5′-end of a second nucleic acid domain has a phosphate group (5′-P).

As discussed above, for the various interactions between the nucleic acid domains to take place, the nucleic acid domains of the proximity probes need to be coupled to the analyte-binding domains in certain orientations. For example, for the extension of both domains wherein said domains comprise single stranded nucleic acids, each nucleic acid domain can be coupled to the analyte-binding domain by its 5′ end, leaving a free 3′ hydroxyl end, which may “anneal” or “hybridise” when in proximity.

For both assays, the nucleic acid domains are generally of a length sufficient to allow interaction with the nucleic acid domain of another proximity probe when bound to a target analyte (or splint-mediated interaction). It is typically from about 20 to about 100 nucleotides in length, from about 20 to about 80 nucleotides in length, from about 25 to about 40 nucleotides in length, from about 25 to about 35 nucleotides in length.

The nucleic domains of the proximity probes must be at least partially single stranded on contact with the sample such that they can interact with each other when binding to the target analyte. Part or whole of this single stranded region can be used as hybridisation region.

### Overlapping/Complementary Sequence

For Proximity Extension Assays, the probes used shall have an hybridisation region that can have a length in the range of 4-30 bp e.g., 6-20, 6-18, 7-15 or 8-12 bp. In a preferred embodiment, the hybridisation regions of the probes of the Proximity Extension Assays have a length from 9 to 11 pb.

### Linking Group

The two components of the proximity probes (nucleic acid domain and analyte-binding domain) can be joined together either directly through a bond or indirectly through a linking group. Where linking groups are employed, such groups may be chosen to provide for covalent attachment of the nucleic acid and analyte-binding domains through the linking group, as well as maintain the desired binding affinity of the analyte-binding domain for its target analyte. Linking groups of interest may vary widely depending on the analyte-binding domain.

The linking group, when present, is in many embodiments biologically inert. A variety of linking groups are known to those of skill in the art and find use in the subject proximity probes. In representative embodiments, the linking group is generally at least about 50 daltons, usually at least about 100 daltons and may be as large as 1000 daltons or larger, for example up to 1000000 daltons if the linking group contains a spacer, but generally will not exceed about 500 daltons and usually will not exceed about 300 daltons.

Generally, such linkers will comprise a spacer group terminated at either end with a reactive functionality capable of covalently bonding to the nucleic acid or analyte binding moieties. Spacer groups of interest may include aliphatic and unsaturated hydrocarbon chains, spacers containing heteroatoms such as oxygen (ethers such as polyethylene glycol) or nitrogen (polyamines), peptides, carbohydrates, cyclic or acyclic systems that may possibly contain heteroatoms. Spacer groups may also be comprised of ligands that bind to metals such that the presence of a metal ion coordinates two or more ligands to form a complex. Specific spacer elements include: 1,4-diaminohexane, xylylenediamine, terephthalic acid, 3,6-dioxaoctanedioic acid, ethylenediamine-N,N-diacetic acid, 1,1 ‘-ethylenebis(5-oxo-3-pyrrolidinecarboxylic acid), 4,4’-ethylenedipiperidine. Potential reactive functionalities include nucleophilic functional groups (amines, alcohols, thiols, hydrazides), electrophilic functional groups (aldehydes, esters, vinyl ketones, epoxides, isocyanates, maleimides), functional groups capable of cycloaddition reactions, forming disulfide bonds, or binding to metals. Specific examples include primary and secondary amines, hydroxamic acids, N-hydroxysuccinimidyl esters, N-hydroxysuccinimidyl carbonates, oxycarbonylimidazoles, nitrophenylesters, trifluoroethyl esters, glycidyl ethers, vinylsulfones, and maleimides.

Specific linker groups that may find use in the subject proximity probes include heterofunctional compounds, such as azidobenzoyl hydrazide, N-[4-(p-azidosalicylamino)butyl]-3′-[2′-pyridyldithio] propionamid), bis-sulfosuccinimidyl suberate, dimethyladipimidate, disuccinimidyltartrate, N-maleimidobutyryloxysuccinimide ester, N-hydroxy sulfosuccinimidyl-4-azidobenzoate, N-succinimidyl [4-azidophenyl]-1,3′-dithiopropionate, N-succinimidyl [4-iodoacetyl]aminobenzoate, glutaraldehyde, and succinimidyl-4-[N-maleimidomethyl]cyclohexane-1-carboxylate, 3-(2-pyridyldithio) propionic acid N-hydroxysuccinimide ester (SPDP), 4-(N-maleimidomethyl)-cyclohexane-1-carboxylic acid N-hydroxysuccinimide ester (SMCC), and the like.

The proximity probes employed in the methods of the invention may be prepared using any convenient method. Methods for producing nucleic acid/antibody conjugates are well known to those of skill in the art. See e.g. U.S. Pat. No. 5,733,523. In other embodiments, proximity probes may be produced using in vitro protocols that yield nucleic acid-protein conjugates, i.e. molecules having nucleic acids, e.g. coding sequences, covalently bonded to a protein, i.e. where the analyte-binding domain is produced in vitro from vectors which encode the proximity probe. Examples of such in vitro protocols of interest include: RepA based protocols (see e.g., WO 98/37186), ribosome display based protocols (see e.g., WO 98/54312), etc.

### Amount

For Proximity Extension Assay and PLA assays, the amount of proximity probes that is added to a sample may be selected to provide a sufficiently low concentration of proximity probe in the reaction mixture to ensure that the proximity probes will not randomly come into close proximity with one another in the absence of binding to a target analyte, at least not to any great or substantial degree.

In representative embodiments, the concentration of Proximity Extension Assay/PLA proximity probes in the reaction mixture following combination with the sample ranges from about 1 μM to 1 μM, such as from about 1 μM to about 1 nM, including from about 1 μM to about 100 nM.

In a particular embodiment, the concentration of PLA/Proximity Extension Assay proximity probes in the reaction mixture ranges from about 200 μM to 800 μM, including from about 250 μM to 500 μM.

### Polymerase

Once the Proximity Extension Assay probes are close-by and hybridize through their oligonucleotide 3′-ends, or once the Proximity Extension Assay extension product is to be detected, or once the circular closed DNA molecules are produced in the PLA assay, addition of a DNA polymerase and nucleotides (dNTPs) to the sample triggers a polymerisation reaction templated either by the nucleic acid domain of the other proximity probe, or by the extension product, or by the circular ligated DNA molecule.

In a particular embodiment, the polymerase used in the PLA Proximity Extension Assay of the invention may be selected in the group consisting of: T4 DNA polymerase, T7 DNA polymerase, Extaq polymerase combined with another enzyme having a 3′>5′ exonuclease activity, Phi29 (@29) DNA polymerase, DNA polymerase I, Klenow fragment of DNA polymerase I, Taq polymerase, KlenTaq polymerase, Pyrococcus furiosus (Pfu) DNA polymerase and/or Pyrococcus woesei (Pwo) DNA polymerase. Depending on the step of the methods of the invention (e.g. extension step, amplification step . . . ), the skilled person is able to select the more suitable polymerase in this list.

In a preferred embodiment, the polymerase used in the PLA/Proximity Extension Assay of the invention is a hyperthermophilic enzyme, particularly a hyperthermophilic polymerase comprising 3′ exonuclease activity, such as Pfu DNA polymerase or Pwo DNA polymerase, having optimum enzymatic activity at above normal biological temperatures, e.g. above 37° C., such as above 40° C., 50° C., 60° C. or 70° C., typically above 60° C. or 70° C.

In a more preferred embodiment, the polymerase used in the PLA/Proximity Extension Assay of the invention is a DNA polymerase selected in the group consisting of: T4 DNA polymerase, T7 DNA polymerase, Extaq polymerase combined with another enzyme having 3′>5′ exonuclease activity, Phi29 (@29) DNA polymerase, DNA polymerase I, Taq polymerase, KlenTaq polymerase and Klenow fragment of DNA polymerase I, and more preferably selected in the group consisting of T4 DNA polymerase and Extaq polymerase combined with another enzyme having 3′>5′ exonuclease activity. In this case, optimum enzymatic activity can be achieved at a temperature comprised between 20° C. and 45° C., typically at 37° C.

In another embodiment, the polymerase used in the PLA/Proximity Extension Assay of the invention is an enzyme with no or minimal 3′ exonuclease activity, e.g. the a subunit of DNA polymerase Ill from E. coli, the Klenow exo (-) fragment of DNA polymerase I, Taq polymerase, Pfu (exo) DNA polymerase, Pwo (exo-) DNA polymerase, KlenTaq polymerase, etc. In a preferred embodiment, in this case, an enzyme comprising 3′ exonuclease activity is added to the sample as a separate entity, preferentially before or contemporaneously with the polymerase required for the extension of the nucleic acid domain, but after the nucleic acid domains of the proximity probes have been allowed to interact, i.e. to form a duplex or to be ligated (cf. WO2012/104261).

When the extension or ligated products are to be amplified, one need to add “amplification reactants” to the sample. By “amplification reactants”, it is herein meant at least primers, polymerase and deoxyribonucleoside triphosphates (dNTPs). They may further include an aqueous buffer medium that includes a source of monovalent ions, a source of divalent cations and a buffering agent. Any convenient source of monovalent ions, such as KCl, K-acetate, NH₄-acetate, K-glutamate, NH₄Cl, ammonium sulphate, and the like may be employed. The divalent cation may be magnesium, manganese, zinc and the like, where the cation will typically be magnesium. Any convenient source of magnesium cation may be employed, including MgCl₂, Mg-acetate, and the like. The amount of Mg²⁺ present in the buffer may range from 0.5 to 10 mM, but will preferably range from about 3 to 6 mM, and will ideally be at about 5 mM. Representative buffering agents or salts that may be present in the buffer include Tris, Tricine, HEPES, MOPS and the like, where the amount of buffering agent will typically range from about 5 to 150 mM, usually from about 10 to 100 mM, and more usually from about 20 to 50 mM, where in certain preferred embodiments the buffering agent will be present in an amount sufficient to provide a pH ranging from about 6.0 to 9.5, where most preferred is pH 7.3 at 72° C. Other agents which may be present in the buffer medium include chelating agents, such as EDTA, EGTA and the like.

### Detection Tools

The amplified products of the final amplification reaction in both assays may be detected using any convenient protocol. Representative non-specific detection protocols include protocols that employ signal producing systems that selectively detect double stranded DNA products, e.g., via intercalation.

Representative detectable molecules that find use in such embodiments include fluorescent nucleic acid stains, such as phenanthridinium dyes, including monomers or homo- or heterodimers thereof, that give an enhanced fluorescence when complexed with nucleic acids. All the detection means described in WO 2012/104261 can be used in the method of the invention, and they do not need to be repeated.

In a preferred embodiment, the extension or ligated product is amplified and detected by quantitative PCR (qPCR), which is also known as real-time PCR. In a particularly preferred embodiment, the qPCR uses a dye which intercalates with nucleic acid molecules to provide a detectable signal, preferably a fluorescent signal. Fluorescent intercalating dyes that may find particular use in the present invention are SYBR Green® and EvaGreen™, although the qPCR embodiments of the invention are not limited to these dyes.

The very final step in the methods of the invention is to detect the signal generated by the labelled amplification products. Signal detection may vary depending on the particular signal producing system employed. In certain embodiments, merely the presence or absence of detectable signal, e.g., fluorescence, is determined and used in the subject assays, e.g., to determine or identify the presence or absence of the target nucleic acid via detection of the pseudotarget nucleic acid and/or amplification products thereof. Depending on the particular label employed, detection of a signal may indicate the presence or absence of the target nucleic acid.

In those embodiments where the signal producing system is a fluorescent signal producing system, signal detection typically includes detecting a change in a fluorescent signal from the reaction mixture to obtain an assay result. In other words, any modulation in the fluorescent signal generated by the reaction mixture is assessed. The change may be an increase or decrease in fluorescence, depending on the nature of the label employed, but in certain embodiments is an increase in fluorescence. The sample may be screened for an increase in fluorescence using any convenient means, e.g., a suitable fluorimeter, such as a thermostable-cuvette or plate-reader fluorimeter. Fluorescence is suitably monitored using a known fluorimeter. The signals from these devices, for instance in the form of photo-multiplier voltages, are sent to a data processor board and converted into a spectrum associated with each sample tube. Multiple tubes, for example 96 tubes, can be assessed at the same time.

When the detection protocol is a real time protocol, e.g., as employed in real time PCR reaction protocols, data may be collected in this way at frequent intervals, for example once every 3 minutes, throughout the reaction. By monitoring the fluorescence of the reactive molecule from the sample during each cycle, the progress of the amplification reaction can be monitored in various ways. For example, the data provided by melting peaks can be analyzed, for example by calculating the area under the melting peaks and these data plotted against the number of cycles. In a preferred embodiment of the invention, the fluorescence signal is achieved using a dye that intercalates in double stranded nucleic acid molecules, preferably wherein the intercalating dye is selected from SYBR Green® and EvaGreen™.

All the details for this final detection step are provided in the art (e.g., in WO 2012/104261 and in WO 2012/160083).

### Description of the Particular Steps of the Invention

As exposed above, the inventors have identified a simpler and shortened Proximity Extension Assay protocol, with a duration reduced by more than 13-folds without significantly affecting the performance of the assay for protein detection, as compared with conventional Proximity Extension Assay protocols. In particular, they show that it is enough to incubate the sample in presence of the probes during a short incubation time, namely between 3-30 minutes, provided that an intermittent shaking is applied. Given the similarities between PLA and Proximity Extension Assay in terms of steps and tools, the same optimized steps are thought to also reduce the time of PLA assays. These PLA and Proximity Extension Assay are commonly referred to as “immuno-molecular assays”.

Therefore, the present invention targets an immuno-molecular assay for detecting a protein analyte (or fragments thereof) in a biological sample, said assay comprising the steps of:
A) Contacting said biological sample with at least two molecules comprising a nucleic acid domain and a binding domain being able to directly or indirectly bind to said protein analyte (or fragments thereof),
B) Incubating the resulting sample from 1 minute to 30 minutes under intermittent shaking for allowing the at least one molecule to bind to the protein analyte,
C) Detecting the binding by a molecular detection method.

In a preferred embodiment, in step B), the sample is incubated from about 3 minutes to about 25 minutes, preferably from about 5 minutes to about 20 minutes, and more preferably from about 5 minutes to about 15 minutes under intermittent shaking.

In a preferred embodiment, the intermittent shaking of step B) comprises alternating periods of shaking lasting from 5 seconds to 30 seconds and static periods lasting from 2 to 3 minutes.

### Application to the Proximity Extension Assays

A general description of Proximity Extension Assays has been provided above, as well as in WO 2012/104261. In Proximity Extension Assays, a pair of antibodies conjugated to unique oligonucleotides (hereafter referred to as “Proximity Extension Assay probes”) binds to their respective epitope. As a result, the probes approach each other by proximity and hybridize through the oligonucleotide 3′-ends. The subsequent addition of DNA polymerase leads to an extension step from this template, creating a DNA duplex that can then be detected and quantified by PCR, preferably by real-time quantitative PCR (qPCR). The signal at the end of the assay is quantitatively proportional to the initial concentration of the target protein.

In a preferred embodiment, the method of the invention is a Proximity Extension Assay, and, in step A) of the method of the invention, the at least one molecule is at least one set of a first and a second proximity probes, each proximity probe comprising an analyte-binding domain and a nucleic acid domain, said analyte-binding domains binding directly to the same protein analyte, and said nucleic acid domains being able to hybridize.

In a preferred embodiment, the Proximity Extension Assay method of the invention contains the following steps I) to VI):
I. Contacting said biological sample with at least one set of a first and a second proximity probes, each proximity probe comprising an analyte-binding domain and a nucleic acid domain, said analyte-binding domains binding to the same analyte, and said nucleic acid domains being able to hybridize,
II. Incubating the resulting sample from 3 to 30 minutes under intermittent shaking, for allowing the proximity probes to bind to the analyte and the nucleic acid domains to hybridize with each other to form a duplex structure,
III. Diluting the resulting mixture sample with a buffer and incubating further the diluted sample,
IV. Adding a DNA polymerase to the sample, and allowing the extension of the 3′ end of at least one nucleic acid domain of said duplex to generate an extension product,
V. Heat inactivating said DNA polymerase,
VI. Detecting the extension product(s), preferably after amplifying them, as described above.

In one embodiment of the Proximity Extension Assay method of the invention, part of the amplification reactants that are used to amplify the extension products in the final step of the Proximity Extension Assay of the invention are the same as those that were used to generate the extension product. In this case, some of the amplification reactants may have been added to the sample following the step III) of incubating the diluted reaction mixture containing proximity probes and the sample. The polymerase should however still be added in the final step, after the inactivation step V). In a preferred embodiment, an aliquot of the sample after step V) is transferred to a new vessel comprising the polymerase, for amplification and detection.

In another embodiment of the method of the invention, the amplification reactants were not added to the sample prior to step VI), and new amplification reactants should be added to the sample at this stage. In a preferred embodiment, an aliquot of the sample after step V) is transferred to a new vessel comprising new amplification reactants (e.g. dNTPs, buffer, polymerase such as Taq polymerase or KlenTaq polymerase), for amplification and detection.

In a preferred embodiment, the polymerase used in step VI) for amplifying the extension products is the Taq polymerase or the KlenTaq polymerase.

### Application to the PLA Assays

A general description of PLA assays has been provided above, as well as in WO 2012/160083. In PLA assays, particular sets of proximity probes are preferably used, containing nucleic acid domains that can be ligated together, i.e., that are each capable of hybridising to each other, so as to bring the respective ends of the probes into juxtaposition for ligation directly or indirectly to one another. Accordingly, the specificity of the ligation reaction is solely dependent upon the single stranded portion of the oligonucleotide part of the proximity probes that have the same sequences.

In a preferred embodiment, the method of the invention is a direct PLA assay, and, in step A) of the method of the invention, the at least one molecule is at least one set of proximity probes, each proximity probe comprising an analyte-binding domain and a nucleic acid domain, said analyte-binding domains binding directly to the same protein analyte, and said nucleic domains being able to be ligated together. According to this embodiment, the PLA assay corresponds to a direct PLA assay.

In a preferred embodiment, the method of the invention is an indirect PLA assay and, in step A) of the method of the invention, the at least one molecule is a first set of at least two proximity probes each comprising an analyte-binding domain and a second set of at least two proximity probes each comprising a binding domain to the first set of proximity probes and a nucleic acid domain, said nucleic domains being able to be ligated together. According to this embodiment, the PLA assay corresponds to an indirect PLA assay.

In a preferred embodiment, the PLA method of the invention comprises the following steps of:
i) Contacting said biological sample with at least one set of two proximity probes, each proximity probe comprising an analyte-binding domain and a nucleic acid domain, said analyte-binding domains binding directly to the same protein analyte, and said nucleic domains being able to be ligated together, or contacting said biological sample with a first set of at least two proximity probes each comprising an analyte-binding domain and with a second set of at least two proximity probes each comprising a binding domain able to bind to at least one probe of the first set of proximity probes and a nucleic acid domain, said nucleic domains being able to be ligated together,
j) Incubating the resulting sample from 1 to 30 minutes under intermittent shaking for allowing the at least one molecule to bind to the protein analyte,
k) Ligating the nucleic acid domains of the proximity probes by a ligase enzyme,
l) Detecting the ligated nucleic acids.

The ligase enzyme that is used in PLA assays is selected on the basis that it cannot ligate mismatched sequences. As is known in the art, ligases catalyze the formation of a phosphodiester bond between juxtaposed 3′-hydroxyl and 5′-phosphate termini of two immediately adjacent nucleic acids when they are annealed or hybridized to a third nucleic acid sequence to which they are complementary (i.e. a template). Any convenient ligase may be employed, where representative ligases of interest include, but are not limited to, temperature sensitive and thermostable ligases. Temperature sensitive ligases include, but are not limited to, bacteriophage T4 DNA ligase, bacteriophage T7 ligase, and E. coli ligase. Thermostable ligases include, but are not limited to, Taq ligase, Tth ligase, and Pfu ligase. Thermostable ligase may be obtained from thermophilic or hyperthermophilic organisms, including but not limited to, prokaryotic, eukaryotic, or archae organisms. Certain RNA ligases may also be employed in the methods of the invention.

All the other useful features and explanations for putting the initial steps i), k) and I) and detecting final steps I) and VI) of the PLA/Proximity Extension Assay methods of the invention into practice have been described above and need not to be repeated here.

All the other steps will be now described in more details below.

### Incubation and Shaking (Step j) of the PLA Assay and Step II) of the Proximity Extension Assay)

Once the sample is contacted with the set(s) of proximity probes, the reaction mixture containing the set(s) of proximity probes and the sample is incubated for a period of time sufficient for the proximity probes to bind target analyte, if present, in the sample.

In a preferred embodiment, the reaction mixture is incubated for a period of time ranging from about 1 minute to about 30 minutes, preferably from about 3 minutes to about 25 minutes, preferably from about 5 minutes to about 20 minutes, preferably from about 5 minutes to about 15 minutes.

As explained in the examples below, the conditions under which the reaction mixture is to be incubated have been optimized by the inventors to promote an efficient and specific binding of the proximity probe to the analyte and appropriate hybridization between the nucleic acid domains carried by the two probes. In particular, it has been surprisingly demonstrated that intermittent shaking was more adapted to favour hybridization than continuous agitation when the incubation time is reduced to 5 minutes. According to the knowledge of the inventors, this is probably because the shearing forces caused by constant shaking did not promote the interaction of ligands to their target.

In a particularly preferred embodiment of the assay of the invention, the reaction mixture is thus incubated for about 5 to 15 minutes.

The present inventors have shown that an incubation procedure of about 10-15 minutes under intermittent shaking is also enough to promote an efficient and specific binding of the proximity probe to the analyte and appropriate hybridization between the nucleic acid domains carried by the two probes (FIG. 3). Therefore, the reaction mixture may be advantageously incubated for about 15 minutes without shaking.

It is also advantageously possible to reduce the incubation procedure to about 5 minutes under intermittent shaking, for example by alternating agitation periods of 10 to 20 seconds and static periods of 1-2 minutes. In this case, preferably, the incubation period of step j)/II) does however not last less than 1 minute, preferably not less than 3 minutes. Surprisingly, the inventors have shown that this intermittent shaking, instead of continuous shaking or without shaking, allows to promote an efficient and specific binding of the proximity probe to the analyte and appropriate hybridization between the nucleic acid domains carried by the two probes (FIG. 3). Therefore, the reaction mixture may be incubated for about 5 minutes under intermittent shaking, alternating agitation periods of 10 to 30 seconds, e.g. 15 seconds, and static periods of 2 to 3 minutes, e.g. 2 minutes and 15 seconds.

In a particular embodiment, the resulting sample is incubated under intermittent shaking from 3 to 25 minutes, preferably from 3 to 20 minutes, more preferably from 3 to 15 minutes.

In another particular embodiment, the resulting sample is incubated under intermittent shaking from 3 to 5 minutes.

By “intermittent shaking”, it is meant that the sample is not agitated constantly, but only during a define period of time, after which it is left static (enduring no agitation) during a define period of time, this cycle of agitation/no agitation periods being repeated during the incubation period. In short, by intermittent shaking, it is also meant a shaking with alternating periods of agitation and stagnation (static period). In the context of the invention, the “agitation” can be performed by mildly rotating the sample (e.g., at about 500, 600, 700 or 800 rpm, maximally 1000 rpm) on a rotative agitator system or by shaking mildly the sample horizontally on a multidimensional agitator system. Preferably, this agitation is performed for a period of time starting from 5 seconds until 1 minute, more preferably from 5 seconds to 30 seconds, even more preferably of about 15s. Then the sample is left static for a period of time from 1 to 10 minutes, preferably from 2 to 5 minutes, more preferably from 2 to 3 minutes.

This incubation step can be performed at a temperature ranging from about 4° C. to about 50° C., including from about 20° C. to about 40° C. Preferably, it is performed at room temperature, i.e., between 20° C. and about 37° C. More preferably, the incubation step is performed at 37° C.

### Dilution+Incubation (Step III) of the Proximity Extension Assay)

Once the sample has been incubated with the proximity Proximity Extension Assay probes in the appropriate conditions explained above, then the sample should be diluted. For example, it is preferred to dilute the sample so that the probes have a final concentration inferior to 100 μM. This dilution step III) in Proximity Extension Assays acts to reduce the possibility of interactions between the unbound proximity probes and/or their interaction with other components in the sample. This dilution step can be performed by adding non-enzymatic components to the mixture reaction, e.g. buffers, salts, nucleotides etc.

As dilution may disrupt the interaction between the nucleic acid domains of the bound probes, it is necessary to let the interaction between the probes stabilise (i.e. re-anneal or re-hybridise) before performing the extension step. This stabilisation can be done by incubating again the diluted sample, for a period of time that is sufficient for the interaction between the bound proximity probes to stabilise. In fact, as shown in the examples below, the present inventors show that it is not necessary to incubate this diluted mixture too long (e.g., for 5 minutes as proposed in the prior art). To stabilise the probes interaction and have a reproducible PCR result at the end of the assay, it is sufficient to let the diluted mixture incubate for a period of time inferior to 5 minutes, preferably from about 20 seconds to about 3 minutes, more preferably from about 20 seconds to about 1 minute. In a more preferred embodiment of the invention, the diluted reaction mixture is incubated for about 30 seconds, so as to stabilize the bound probes after dilution occurred.

This second incubation step III) of the Proximity Extension Assays can be performed at a temperature ranging from about 4° C. to about 50° C., including from about 20° C. to about 40° C. Preferably, this second incubation step is performed at about 37° C.

Extension with DNA Polymerase (Step IV) of the Proximity Extension Assay)

Further to these dilution steps, in the Proximity Extension Assays, the sample is contacted with the DNA polymerase enzyme and appropriate buffers and/or other salts/components that are required for the generation of the extension product.

Optionally, a component comprising 3′ exonuclease activity can be added before or contemporaneously with the DNA polymerase enzyme required for the extension reaction. In a preferred embodiment, the polymerase enzyme comprises also 3′ exonuclease activity. The sample is then further incubated under the appropriate conditions to allow the 3′ exonuclease activity to act on the nucleic acid domains of unbound proximity probes and/or the DNA polymerase enzyme to extend the hybridized region of the nucleic acid domains of the probes.

In embodiments in which the polymerase used to extend the nucleic acid domains is a hyperthermophilic polymerase as defined above. The skilled person is capable of adapting the temperature for the extension reaction and, if the polymerase also has 3′ exonuclease activity, the exonuclease reaction, depending on the enzyme's characteristics which are well known. For example, the temperature may range from about 40° C. to about 80° C., including from about 40° C. to about 75° C., from about 40° C. to about 75° C., or from about 50° C. to about 75° C. or from about 68° C. to about 72° C.

Alternatively, if the polymerase used to extend the nucleic acid domains is not thermostable, then it should be kept at a temperature comprised between about 20° C. to about 45° C., typically at 37° C.

Preferably, the extension reaction lasts for no more than 5 minutes, preferably for no more than 3 minutes, more preferably for no more than 2 minutes. In a preferred embodiment, the extension reaction lasts for only 1 minute.

The preferred polymerase used in step IV) of the Proximity Extension Assay according to the invention is a polymerase having a 3′>5′ exonuclease activity, and is advantageously selected from the group consisting of: T4 DNA polymerase, T7 DNA polymerase, Extaq polymerase combined with another enzyme having a 3′>5′ exonuclease activity, and more preferably the polymerase is the T4 DNA polymerase. The extension reaction with this preferred T4 DNA polymerase lasting for about 1 minute, at 37° C.

### Inactivation of the Enzymes (Step V) of the Proximity Extension Assay)

Following the generation of the extension products during step IV) of the Proximity Extension Assay, the polymerase and/or the component comprising 3′ exonuclease activity may be inactivated. In a preferred embodiment, this inactivation is performed by heat denaturation, e.g. by submitting the sample to high temperature (typically from 65° C. to 90° C., preferably 80° C.).

As apparent in the examples below, the present inventors show that it is not necessary to inactivate the enzymes for too long (e.g., for 10 minutes as proposed in the prior art). It is in fact sufficient to inactivate the enzymes for a short time, e.g., from 10 seconds to 3 minutes, preferably from 20 seconds to 1 minute, typically during 30 seconds.

Taking all these embodiments into account, the total duration of the steps of the assay of the invention (without considering the detection step VI) of the Proximity Extension Assay) is shortened to about 10, preferably to 7 minutes, without the sensitivity of the detection to be affected.

### Detecting the Extension Product (Step VI) of the Proximity Extension Assay)

As mentioned above, the detection protocol can include an amplification reaction in which the copy number of the extension product nucleic acid (or part thereof) is increased. As also mentioned above, the extension product can be detected by any amplification method known by the skilled person in the art. The amplification may be linear or exponential, as desired, where representative amplification protocols of interest include, but are not limited to: polymerase chain reaction (PCR); isothermal amplification, Rolling circle amplification, etc. All these protocols are well-known in the art.

In a preferred embodiment, the final step of the Proximity Extension Assay according to the invention consists in amplifying the extension product by PCR or qPCR, so as ensure that it can be detected (its existence meaning that the analyte was present in the sample).

The term “amplifying” is used generally herein to include any means of increasing the number of copies of the extension product or part thereof. Any amplification means known in the art may be utilised in the methods of the invention, e.g. PCR, LCR, RCA, MDA etc. Depending on the abundance of the target analyte in the sample, it may be necessary to amplify the extension product, or part thereof, such that the concentration of the extension product has doubled, i.e., 2 times the number of copies present before amplification. Alternatively, it may be preferable to increase the number of copies by multiple orders of magnitude. In some embodiments amplification results in the sample comprising at least 2, 3, 4, 5, 6, 7, 8, 9, 10, 15, 20, 50 or 75 times the original amount of extension product or part thereof. In further preferred embodiments it may be preferable to amplify the extension product such that the sample comprises at least 102, 103, 104, 105, 106, 107, 108, 109, 1010 etc. times the original amount of extension product or part thereof.

This amplification is preferably achieved by using a Taq polymerase or a KlenTaq polymerase.

### Definitions

As used herein, the expressions “immuno-molecular assay” or “immuno-molecular method” are synonyms and refer to assays or methods that combine the immunocapture of an analyte (e.g. by at least a proximity probe) and a molecular amplification method (e.g. PCR or qPCR), for highly sensitive and selective detection of said analyte. Examples of well-known immuno-molecular assays are Proximity Ligation Assay, Immuno-PCR or Proximity Extension Assay.

As used herein, the term “detecting” includes determining, measuring, assessing or assaying the presence or absence or amount or location of analyte in any way. Quantitative and qualitative determinations, measurements or assessments are included, including semi-quantitative. Such determinations, measurements or assessments may be relative, for example when two or more different analytes in a sample are being detected, or absolute. As such, the term “quantifying” when used in the context of quantifying a target analyte(s) in a sample can refer to absolute or to relative quantification. Absolute quantification may be accomplished by inclusion of known concentration(s) of one or more control analytes and/or referencing the detected level of the target analyte with known control analytes (e.g., through generation of a standard curve). Alternatively, relative quantification can be accomplished by comparison of detected levels or amounts between two or more different target analytes to provide a relative quantification of each of the two or more different analytes, i.e., relative to each other.

As used herein, an “analyte” may be any substance (e.g. molecule) or entity it is desired to detect in a sample. The analyte is the “target” of the assay method of the invention. The analyte may accordingly be any biomolecule or chemical compound it may be desired to detect, for example a peptide or protein. The analyte may be a cell or a microorganism, including a virus, or a fragment or product thereof. It will be seen therefore that the analyte can be any substance or entity for which a specific binding partner (e.g., an affinity binding partner) can be developed. All that is required is that the analyte is capable of simultaneously binding at least two binding partners (more particularly, the analyte-binding domains of at least two proximity probes). Analytes of particular interest include proteinaceous molecules such as peptides, polypeptides, proteins or prions or any molecule which includes a protein or polypeptide component, etc., or fragments thereof. The analyte may be a single molecule or a complex that contains two or more molecular subunits, which may or may not be covalently bound to one another, and which may be the same or different. Thus in addition to cells or microorganisms, such a complex analyte may also be a protein complex. Such a complex may thus be a homo- or hetero-multimer. Aggregates of molecules e.g. proteins may also be target analytes, for example aggregates of the same protein or different proteins. The analyte may also be a complex between proteins or peptides and nucleic acid molecules such as DNA or RNA. Of particular interest may be the interactions between proteins and nucleic acids, e.g. regulatory factors, such as transcription factors, and DNA or RNA.

As used herein, the term “sample” includes any biological and clinical samples, e.g. any cell or tissue sample of an organism, or any body fluid or preparation derived therefrom, as well as samples such as cell cultures, cell preparations, cell lysates etc. Environmental samples, e.g., soil and water samples or food samples are also included. The samples may be freshly prepared or they may be prior-treated in any convenient way, e.g. for storage. The sample may be a biological sample, which may contain any viral or cellular material, including all prokaryotic or eukaryotic cells, viruses, bacteriophages, mycoplasmas, protoplasts and organelles. Such biological material may thus comprise all types of mammalian and non-mammalian animal cells, plant cells, algae including blue-green algae, fungi, bacteria, protozoa etc. Representative samples thus include whole blood and blood-derived products such as plasma, serum and buffy coat, blood cells, urine, faeces, cerebrospinal fluid or any other body fluids (e.g. respiratory secretions, saliva, milk, etc), tissues, biopsies, cell cultures, cell suspensions, conditioned media or other samples of cell culture constituents, etc.

The sample may be pre-treated in any convenient or desired way to prepare for use in the method of the invention, for example by cell lysis or purification, isolation of the analyte, etc.

A typical volume of the reaction mixture is of 4 μL to which 4 μL of a buffer containing the probes is added.

In certain embodiments, a sample may be assayed for two or more different target analytes. In such embodiments, the sample is contacted with a set of proximity probes for each target analyte, such that the number of sets contacted with the sample may be two or more, e.g., three or more, four or more etc. Such methods find particular use in multiplex and high-throughput applications.

### Possible Improvements

Crowding agent

In certain embodiments, the effective volume of the incubation mixture is reduced, at least during the portion of the incubation step in which the proximity probes are binding to target analyte, if present in the sample. In these embodiments, the effective volume of the incubation mixture may be reduced for a number of different reasons, e.g., in order to allow for the use of medium and low affinity analyte-binding domains and/or increase the sensitivity of the assay. For example, in certain embodiments where the effective volume of the incubation mixture is reduced, the analyte-binding domains may be medium or low affinity binders, by which is meant that the analyte-binding domains may have a binding affinity for their target analyte that is less than about 104 M, such as about 1 nM Kd. In certain embodiments, the sensitivity of the assay may be increased such that the assay can detect as few as about 100 or fewer target analytes in a 1 μl sample, including as few as about 75 or fewer target analytes in a 1 μl sample, including as few as about 50 or fewer target analytes in a 1 μl sample.

### Splint

To reduce non-specific background signal, it is possible to add in the sample of the invention a splint oligonucleotide which is the nucleic acid domain of a third proximity probe (WO 2007/107743). This can improve the sensitivity and specificity of the assay.

### Blocking Agents

Other methods for reducing non-specific background signal have been proposed, such as using blocking reagents, e.g. blocking oligonucleotides, which bind to the free ends of the nucleic acid domains on the proximity probes until displaced by, e.g. a displacer oligonucleotide. Such a displacer oligonucleotide is allowed to bind to the analyte only after the proximity probes, so that interaction of the nucleic acid domains of the proximity probes is likely to occur only for the proximity probes bound to the target analyte (WO 2007/107743).

### Ligation (for Proximity Extension Assays)

In some embodiments of the Proximity Extension Assay, the nucleic acid domains of the Proximity Extension Assay probes may be ligated together, following the extension of one of the nucleic acid domains, to produce the extension products. In embodiments where the nucleic acid domains are ligated, this ligation is typically mediated by a splint, which may be considered to be part of the nucleic acid domain of one of the proximity probes, i.e., wherein the nucleic acid domain is partially double stranded. The splint may be provided separately, either as a free nucleic acid molecule or it can be provided as the nucleic acid domain of a third proximity probe. The ligation results in the formation of a new nucleic acid molecule or sequence, which may then be amplified and detected (WO 2012/104261).

### Immobilization on an Array

Of particular interest is the combination of the present method with a “DNA array” read-out format. Several unique extension products from a multiplexed proximity extension assay as described herein may be hybridized to a standardized DNA array carrying a number of oligonucleotide sequences (tags) complementary to the extension product sequences. Each extension product hybridized to the array may be identified by its location on the DNA array and the detected intensity in a given hybridization spot will be indicative of the quantity of that specific extension product and hence also of the analyte giving rise to that extension product. Detection of the extension products may be accomplished by spectrometry, fluorescence, radioisotopes etc. Fluorescent moieties may conveniently be introduced into the extension products using fluorescently labelled primers or fluorescently labelled nucleotides in the amplification reaction (PCR). The DNA array may be a simple dot-blot array on a membrane containing a small number of spots or a high density array carrying hundreds of thousands of spots (WO 2012/104261).

### Proof of Concept: Detection of PCT

As an initial proof of concept, the inventors have set up an optimized Proximity Extension Assay protocol to detect physiological amounts of the protein procalcitonin (PCT) in very small volumes of human plasma samples.

PCT is used in clinical practice for improved patient management for suspected bacterial infection. It is a 14.5 kDa polypeptide which is stored, after its synthesis, in secretory granules in all cell types of the body. PCT is a pro-hormone whose blood level rises and can be routinely measured early and specifically in the case of bacterial infection but also in the case of fungal or parasitic infection. PCT is a versatile biological marker that can be used to assist in the etiological diagnosis of an infection, help determine its severity, monitor its evolution and response to treatment, and adapt the treatment for each patient [16, 17]. It is generally accepted that for values below 0.25 μg/L a bacterial infection is excluded. A plasma dose above 0.5 μg/L indicates local infection, or even systemic bacterial infection for a dose above 2.0 μg/L. At a dose of 10 μg/L or more, a diagnosis of severe sepsis and/or septic shock can be made [19].

The examples below show that the short Proximity Extension Assay of the invention enables quantification of PCT over a large range of concentrations and with low limit of detection (0.1 ng/mL) in a human plasma.

In particular, the examples below show a Proximity Extension Assay method containing the following steps:
a) Contacting a plasma sample with at least one set of a first and a second proximity probes, each proximity probe comprising an anti-PCT antibody domain and a nucleic acid domain, said anti-PCT antibody domains binding to PCT, and said nucleic acid domains being able to hybridize, b) Incubating the resulting sample for 15 minutes under intermittent shaking,
c) Diluting the resulting mixture sample with a buffer and incubating further the diluted sample for 30 seconds at 37° C.,
d) Adding the T4 DNA polymerase to the sample, and allowing the extension of the 3′ end of at least one nucleic acid domain of said duplex to generate an extension product for 1 minute at 37° C.,
e) Heat inactivating said DNA polymerase for 30 seconds at 80° C.,
f) Amplifying and detecting the extension product(s).

The antibodies that can be used are monoclonal antibodies 6H11 and 8F12 (anti-calcitonin), associated to monoclonal antibody 11E12 (anti-katacalcin). All these antibodies may be provided by BioMérieux upon request.

The sequences of the oligonucleotides of the MM probes are given below (probe MM7):
Oligo1: TAATAGTATCGAGCGGTCAGAGACGACTTC (SEQ ID NO:1)
Oligo2: TAGCTAAGTGGCAGGATGAGAAGTCGTC (SEQ ID NO:2)

### FIGURE LEGENDS

FIG. 1 discloses the selection of monoclonal antibody pair for Proximity Extension Assay PCT. (A) OD at 405 nm for the different PCT antibody pairs tested in ELISA. For each pair, antibodies were tested either as capture antibody (c) or as detection antibody (d). Different incubation times were evaluated (60, 30, 15, 5, 1 and “0” minutes) for monoclonal antibodies binding to rPCT. The results correspond to the average of three duplicate experiments performed for each condition, after subtracting the blank (assay without rPCT). The black line corresponds to a positivity threshold representing 3 times the OD signal obtained on the blank. (B) Proximity Extension Assay performed using different pairs of PCT monoclonal antibodies. Antibodies were 11E12 (present in all conditions) associated to either 6H11, 8F12, 4C10, 5G5 or 11H4. Results are given in Cq (cycle of quantification) values, which corresponds to the smallest qPCR cycle number at which the fluorescence signal is significantly higher than the background. The data are also expressed in Delta-Cq (table below) corresponding to the difference between the Cq value obtained for 0 ng/mL of rPCT and the Cq value obtained for different concentrations of rPCT (0.1, 1.5 and 10 ng/ml, respectively). The results correspond to the mean of four duplicate experiments.

FIG. 2 discloses Proximity Extension Assayperformed for different durations of antigen/antibody binding, for three concentrations of rPCT. The time of incubation between the rPCT and the Proximity Extension Assay probes was progressively decreased from 1h (reference test, ref.) down to 5 minutes. Incubation was performed at 37° C. under static conditions (no agitation). The results, given in Cq, correspond to the mean of a minimum of three duplicate experiments. The data are also expressed in Delta-Cq (table below) corresponding to the difference between the Cq value obtained for 0 ng/ml of rPCT and the Cq value obtained for different concentrations of rPCT.

FIG. 3 discloses the effect of plate agitation on Proximity Extension Assayperformed for different durations of antigen/antibody binding for three concentrations of rPCT. The incubation time between the rPCT and the Proximity Extension Assay probes was progressively decreased from 1h (reference test) down to 5 minutes. The incubation was performed at 37° C. under static conditions (static) or with agitation of the multi-well plate in a thermomixer. Two alternative mixing conditions were tested: one with continuous agitation at 500 rpm, the other with phases of agitation at 800 rpm alternating with phases without agitation (static). The results, given in Cq values, correspond to the mean of a minimum of three duplicate experiments. The data are also expressed in Delta-Cq (table below) corresponding to the difference between the Cq value obtained for 0 ng/ml of rPCT and the Cq value obtained for different concentrations of rPCT.

FIG. 4 shows the result of the Proximity Extension Assayperformed at different times of incubation after the dilution step, for three concentrations of rPCT. The time of incubation after dilution of the binding product was progressively decreased from 5 min (reference test, ref.) down to 30 sec. The results, given in Cq values, correspond to the mean of a minimum of three duplicate experiments. The data are also expressed in Delta-Cq (table below) corresponding to the difference between the Cq value obtained for 0 ng/mL of rPCT and the Cq value obtained for different concentrations of rPCT.

FIG. 5 shows the result of the Proximity Extension Assayperformed at different extension times and different enzyme inactivation times, for three concentrations of rPCT. The time of extension (Ext.) of the duplex by T4 DNA polymerase was progressively decreased from 20 min (reference test, ref.) down to 1 min. In parallel, the time of inactivation of the enzyme (inact.) was reduced from 10 min (reference, ref.) down to 30 sec at 80° C. The results, given in Cq values, correspond to the mean of a minimum of three duplicate experiments. The data are also expressed in Delta-Cq values (table below) corresponding to the difference between the Cq value obtained for 0 ng/ml of rPCT and the Cq value obtained for different concentrations of rPCT.

FIG. 6 shows the result of short Proximity Extension Assayprotocol implementation in its full form on rPCT spiked in PBS-BSA buffer. The short protocol was compared in terms of Cq values with the reference protocol, on three rPCT concentrations. The results, given in Cq, correspond to the mean of a minimum of three duplicate experiments. The data are also expressed in delta Cq (table below) corresponding to the difference between the Cq value obtained for 0 ng/ml of rPCT and the Cq value obtained for different concentrations of rPCT.

FIG. 7 shows the comparison of different enzymes or enzyme combinations for Proximity Extension Assay extension step (short protocol), for three concentrations of rPCT. T4: T4 DNA polymerase; Extaq: Extaq polymerase; Exo I: exonuclease |; Exo VII: exonuclease VII; Exo T: exonuclease T. The results, given in Cq, correspond to the mean of a minimum of three duplicate experiments. The data are also expressed in delta Cq (table below) corresponding to the difference between the Cq value obtained for 0 ng/ml of rPCT and the Cq value obtained for different concentrations of rPCT.

FIG. 8 shows the result of the short Proximity Extension Assayprotocol implemented on human plasma specimens containing different concentrations of PCT. The results, given in Cq values, correspond to the mean of two or three duplicate experiments. The grey star corresponds to an estimation of the LoD.

### EXAMPLES

### Material and Methods

### Material

Recombinant human procalcitonin (rPCT) was developed and produced by bioMérieux SA (Lyon, France) after protein expression in prokaryotic cells, according to standard procedures [20]. rPCT was stored at −80° C. in a PBS buffer containing 5% bovine serum albumin (BSA).

Whole blood specimens were obtained from healthy donors from the French National Blood Bank (Etablissement Français du Sang Auvergne-Rhône Alpes (EFS), Décine Charpieu, France). Specimens were selected on the basis of a negative PCT test. Fresh blood was centrifuged for five min at 20 000 g, at 4° C., using a refrigerated microcentrifuge (Eppendorf 5424R, Montesson, France). After centrifugation, the supernatant, which corresponds to the plasma, was recovered for further analysis.

A set of PCT-positive EDTA plasma samples (leftovers) was obtained from the Saint Luc-Saint Joseph Hospital (Lyon, France) through specific supply agreements with bioMerieux SA. The PCT concentration in each of these samples determined using a commercial IVD test (BRAHMS Vidas PCT, BioMerieux) ([21]).

For all biological samples, informed consent was obtained prior to any experimentation. All experiments were performed in compliance with the relevant laws and institutional guidelines and in accordance with the ethical standards of the Declaration of Helsinki. A framework agreement (“convention of session of products derived from human blood for non-therapeutic purposes”) has been established between BioMerieux SA and the French National Blood Bank for two years and is renewable. EFS is in charge of the ethic approval for all samples. The current agreement (EFS20-294) has been established in December 2020.

Monoclonal antibodies were produced by bioMerieux SA according to standard procedures, following mice immunizations with internally produced recombinant PCT and are non-commercial antibodies. These antibodies were directed either against the Calcitonin domain of the PCT molecule (monoclonal antibodies 11H4, 5G5, 4C10, 6H11 and 8F12), or against the Katacalcin domain (monoclonal antibody 11E12). A biotin-labelled version of these antibodies was also prepared for use in sandwich ELISA (Enzyme Linked Immuno-Sorbant Assay).

For Proximity Extension Assay development, two DNA polymerases were evaluated: T4 DNA polymerase (ThermoFisher Scientific, Waltham, MS, USA) and Extaq polymerase (Takara Bio, Kusatsu, Japan). Several 3′>5′ exonucleases were also tested: exonuclease I, exonuclease VII, and exonuclease T (all from New England Biolabs, Ipswich, MS, USA).

### Proximity Extension Assay Probes

Proximity probes were prepared by covalently linking purified PCT monoclonal antibodies to HPLC-purified oligonucleotides having a C6-amin modification at the 5′ end. Synthesis, derivatization and HPLC-purification of the oligonucleotides were all performed by Integrated DNA Technologies (Coralville, USA). Antibody-oligonucleotide conjugates were prepared using commercial Thunderlink kit (Abcam, Cambridge, UK), following the manufacturer's instructions, with a 5:1 oligonucleotide-to-antibody ratio. Conjugation quality was assessed by running conjugates on a reducing 4-12% SDS-PAGE (Thermofisher) followed by Coomassie Blue staining (ThermoFisher Scientific) as recommended by the supplier's instructions (Abcam). The oligonucleotide comprised at least, from 5′ to 3′, a 18-22 nucleotides sequence for PCR primer hybridization and a 9-12 nucleotides sequence for hybridization with the second Proximity Extension Assay probe. Sequences were generated randomly (https://molbiotools.com), taking care to minimize homeo-tracks and palindromic sequences. The PCR primers sequences had a Tm around 60° C. For the hybridization zone, a % CG between 50 and 60% was preferred.

### Sandwich ELISA

After overnight sensitization with monoclonal antibodies diluted at 5 μg/mL in PBS buffer, pH 7.4 (Euromedex, Souffelweyersheim, France), Nunc 96-well plates (ThermoFisher Scientific) were saturated for 1 h at room temperature (RT) with PBS1x-Bovine Serum Albumin 5% (BSA, Sigma-Aldrich, Saint-Louis, Mi USA). After 4 washes with PBS1x-Tween20 0.1%, plasma containing (or not) the rPCT protein (10 ng/ml) plus a biotin-labelled antibody (1 μg/mL) were added to the plate and incubated for 1 h at 37° C.

The developing with PNPP substrates (ThermoFisher Scientific) was performed after 30 min of incubation at 37° C. with a 1/50 000 dilution in PBS-BSA-0.5% of an alkaline phosphatase (AP)-labeled streptavidin (Jackson Immunoresearch, Ely, UK). The absorbance level (optical density, OD) was read at 405 nm (OD405) using an Infinite M Nano+microplate reader (Tecan). All samples were tested in duplicate and in two independent experiments.

### Proximity Extension Assay

The Proximity Extension Assay reference protocol and the various buffer formulations were taken over from reference [8].

Proximity Extension Assay was performed in specific multi-well plates (Hard-Shell 96-Well Clear shell PCR Plates, BioRad, Hercule, CA USA), in duplicate. Briefly, 4 μL of sample (PBS1x-0.1% BSA buffer with or without rPCT or human EDTA plasma) were combined with 4 μL of a mix of the two Proximity Extension Assay conjugates (at 500 μM each) diluted in probe incubation buffer and incubated at 37° C. for 1 h. After probe incubation, 96 μL of dilution buffer containing 40 mM of each dNTP were added. After a 5-min incubation step at 37° C., 96 μL of extension mix containing 26 U/mL of T4 DNA Polymerase (ThermoFisher Scientific) were added and incubated at 37° C. for another 20 min, followed by a 10-min heat inactivation step at 80° C.

Detection by qPCR

For qPCR detection, 4 μL of Proximity Extension Assay extension products were transferred to a qPCR plate and mixed with 36 μL of qPCR mix (SYBRGREEN mix, Bio-Rad) containing 0.6 UM of each PCR primer. One-step qPCR was run with initial denaturation at 95° C. for 3 min, followed by 1 sec of denaturation at 95° C. and 10 sec of annealing/extension at 63° C. for 40 cycles. Finally, a melt step was performed by increasing the temperature gradually from 60° C. to 95° C. at 0.5° C./sec.

### Statistical Analysis

Means and standard deviations were calculated using Excel 2019 (Microsoft, Albuquerque NM USA).

### Results

### Antibody Selection

Proximity Extension Assay requires the use of two specific antibodies. To minimize assay time, antibodies must exhibit high affinity to the PCT molecule. Moreover, these two antibodies must not interfere with each other upon binding to the analyte, i.e., they must not target the same region of the protein. PCT is a small (14.5 kDa) polypeptide consisting of two domains: the calcitonin domain, located in the N-terminal region of the PCT molecule, and the katacalcin domain, which constitutes its C-terminal part. In order to limit binding interference, it was decided to select one antibody that targets calcitonin and another one that targets katacalcin. Another advantage of this approach is that PCT metabolic by-products like plasma calcitonin or katacalcin are not detected.

Proximity Extension Assay can be considered to be a sandwich assay. In order to choose the best pair of antibodies in terms of compatibility and affinity (more specifically, the on-rate constant (K-on) should be as high as possible), different pairs of antibodies were evaluated in a sandwich ELISA format.

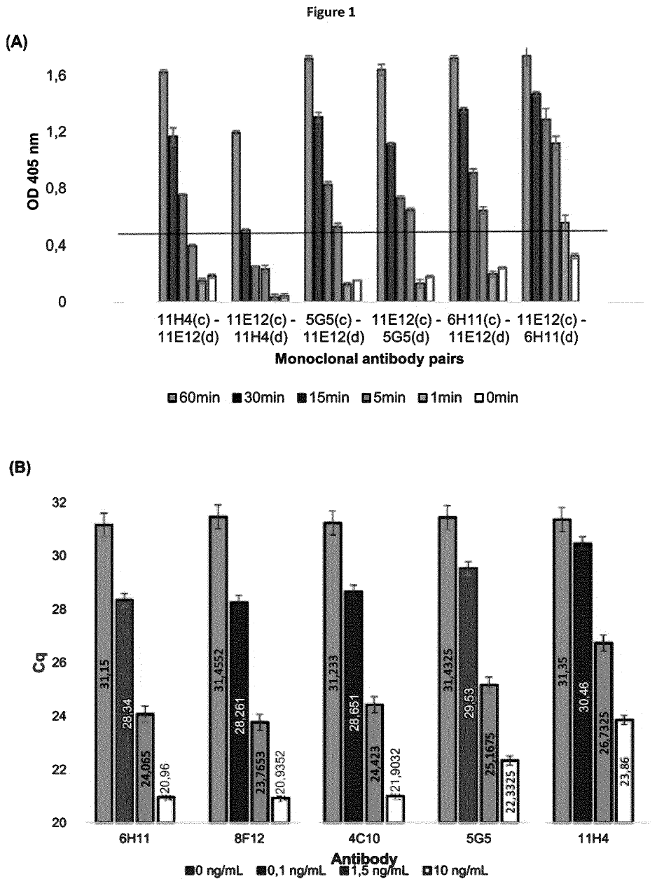

Each antibody was alternatively used either as a capture antibody (after coating on the well of the plate) or as a detection antibody (free in solution). First, these pairs were evaluated using different binding times between the antibodies and the rPCT (from 0 min to 60 min) before proceeding to the signal revelation step. Note that for the “zero” minute condition, the mix containing rPCT and detection antibody was added to the wells and immediately removed by washing. As illustrated in FIG. 1A, a 60 min of incubation, which corresponds to the time dedicated to the antigen/antibody binding in the reference protocol, all pairs except 11H4/11E12 performed identically, with an OD405 around 1.6. In contrast, for less than 5 min of incubation, the signal remained under the threshold defined as 3 times the background level (OD405=0.43), except for the 11E12-capture/6H11-detection pair [see FIG. 1A: 11E12 (c)-6H11 (d)]. For 5, 15 and 30 min, the highest signal was obtained for the 11E12/6H11 pair. For 15 min of incubation, OD405=0.91+/−0.05 [6H11 (c)-11E12 (d)] and OD405=1.29+/−0.08 [11E12 (c)-6H11 (d)].

In order to confirm these results in the Proximity Extension Assay format, experiments were conducted using the above antibodies (evaluated in ELISA), plus two additional antibodies called 8F12 and 4C10. All these antibodies were conjugated to oligonucleotides as described above. FIG. 1B shows the Cq values obtained for four concentrations of rPCT (10 ng/ml, 1.5 ng/ml, 0.1 ng/ml and 0 ng/ml. As expected, the Cq value decreased when rPCT concentration increased. An equivalent background noise (31.15<Cq0 ng/mL<31.45) was observed for all the antibodies. However, for 0.1, 1.5 and 10 ng/ml of rPCT, the Cq was mostly lower for 6H11 and 8F12 antibodies than the other anti-calcitonin antibodies (specifically, 5G5 and 11H4).

TABLE 1

| Delta-Cq | 6H11 | 8F12 | 4C10 | 5G5 | 11H4 |
|---|---|---|---|---|---|
| 0-0.1 | 2.81 | 3.19 | 2.58 | 1.9 | 0.89 |
| 0.1.5 | 7.09 | 7.69 | 6.81 | 6.2 | 4.62 |
| 0-10 | 10.19 | 10.52 | 10.23 | 9.1 | 7.5 |

These results confirmed that monoclonal antibodies 6H11 and 8F12 (anti-calcitonin), associated to 11E12 (anti-katacalcin), gave the higher sensitivity for the three rPCT concentrations tested. Results also confirmed that the 5G5/11E12 and 11H4/11E12 pairs of antibodies were less efficient. For the 6H11/11E12 and the 8F12/11E12 pairs, Delta-Cq0-10 was around 1.4 higher than for the 11H4/11E12 pair. For Delta-Cq0-0,1, it was 2.9-3.5 higher. Consequently, 6H11 (and 8F12) and 11E12 were selected for further investigations.

### Probe Selection

Regarding probes synthesis, successive versions of oligonucleotides pairs were evaluated. Because no complete 3D-model of PCT molecule the protein structure was available, it was not possible to determine the distance between epitopes where antibodies bind. To assess whether long oligonucleotides were needed for the PCT Proximity Extension Assay (as would be the case if the antibodies bind far from each other), the initial pair of oligonucleotides tested (MM1) was designed with 62-90 nucleotides (nt) sequences and a 12-mer hybridization zone. Each oligonucleotide included sequences for PCR primers hybridization in addition to the hybridization zone, separated by junk sequences. Because these junk sequences can eventually form 2D structures or facilitate unwanted hybridizations with unfavourable impact on the processivity of polymerases, some of these junk sequences were removed in the next versions of the probe (MM2 and MM4) and the hybridization zone was gradually reduced. Finally, in an effort to simplify the oligonucleotides as much as possible, the junk sequences were totally removed in the ultimate versions (MM6 to MM9), and the hybridization zone continued to be downsized. The characteristics of these successive versions, and the associated performances obtained in Proximity Extension Assay, are illustrated in Table 2.

TABLE 2
Main characteristics of the oligonucleotide sets used
for Proximity Extension Assay for PCT detection.

| Name of | Oligonucleotide | Hybridation Zone | | Highest Delta-Cq₀₋₁₀ |
|---|---|---|---|---|
| probes | sizes | (HZ) size | HZ % CG | using PEA |
| MM1 | 90 nt | 12 nt | 58.3% | 3.07 |
| | 62 nt | | | |
| MM2 | 51 nt | 12 nt | 58.3% | 3.35 |
| | 59 nt | | | |
| MM4 | 51 nt | 11 nt | 54.5% | 9.83 |
| | 59 nt | | | |
| MM6 | 33 nt | 11 nt | 54.5% | 9.98 |
| | 32 nt | | | |
| MM7 | 31 nt | 10 nt | 50% | 12.2 |
| | 29 nt | | | |
| MM9 | 30 nt | 9 nt | 55% | 11.9 |
| | 28 nt | | | |

nt: nucleotides; Delta-Cq₀₋₁₀: difference of Cq between 0 and 10 ng/mL of PCT.

The shorter the hybridization zone, the higher the Delta-Cq (0-10). The size of the oligonucleotides seemed to have little impact since the Delta-Cq between MM4 and MM6 was similar (9.83 vs 9.98). In addition, the difference observed between the Delta-Cq0-10 of MM2 (3.35) and MM4 (9.83) can only be attributed to the size of the hybridization zone.

In conclusion, pairs of monoclonal antibodies and oligonucleotides were selected to prepare probes (proximity probes) specifically adapted to the development of an efficient PCT Proximity Extension Assay. The pair of Proximity Extension Assay probes used for the rest of the study were thus prepared from the 6H11 and 11E12 monoclonal antibodies conjugated to the MM7 oligonucleotides.

### Reduction of the Proximity Extension Assay Time Experiment

In order to dramatically reduce the duration of the assay while maintaining a high level of performance, a gradual decrease in incubation times was assessed for each step of the assay. This was first conducted separately and then evaluated in a single merged protocol.

Conventionally, in the reference protocol, the time required for the binding of the antigen to the Proximity Extension Assay conjugates is about one hour. Different incubation times, including 15 min and 5 min, were tested for three concentrations of rPCT (0, 1.5 and 10 ng/mL) diluted in PBS1x-BSA. These rPCT concentrations correspond to concentrations of PCT found in patients with systemic bacterial infections (1.5 ng/ml) or severe sepsis (10 ng/ml). No agitation of the plates was provided during incubation, which took place in static conditions.

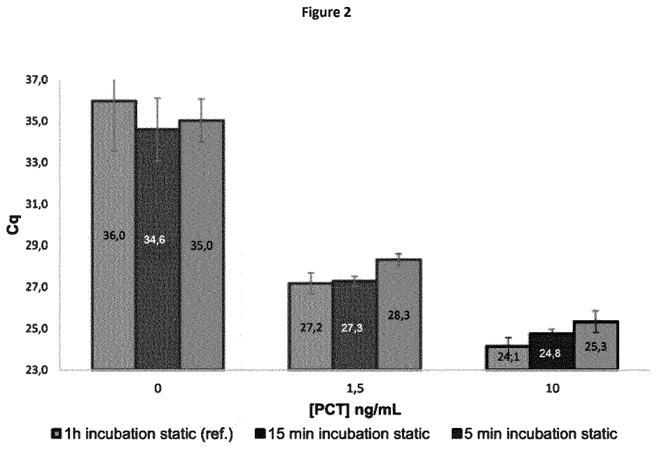

As illustrated in FIG. 2, using the pair of Proximity Extension Assay probes previously selected, the Cq value obtained with 1 h or 15 min of incubation was not significantly different for the two concentrations of rPCT. However, at 5 min of incubation a slight but significant loss of sensitivity was observed for 1.5 and 10 ng/ml of rPCT.

Results expressed in Delta-Cq also showed that for 1.5 ng/ml of rPCT, with 15 min or 5 min of incubation, the differences of Delta-Cq (Delta [Delta-Cq]) for 1 h of incubation was 1.5 and 2.1, respectively. For 10 ng/ml of rPCT, the Delta [Delta-Cq] was 2.1 and 2.2, respectively.

TABLE 3

| Delta Cq | 1 h static (ref.) | 15 min static | 5 min static |
|---|---|---|---|
| 0-1.5 | 8.8 | 7.3 | 6.7 |
| 0-10 | 11.9 | 9.8 | 9.7 |

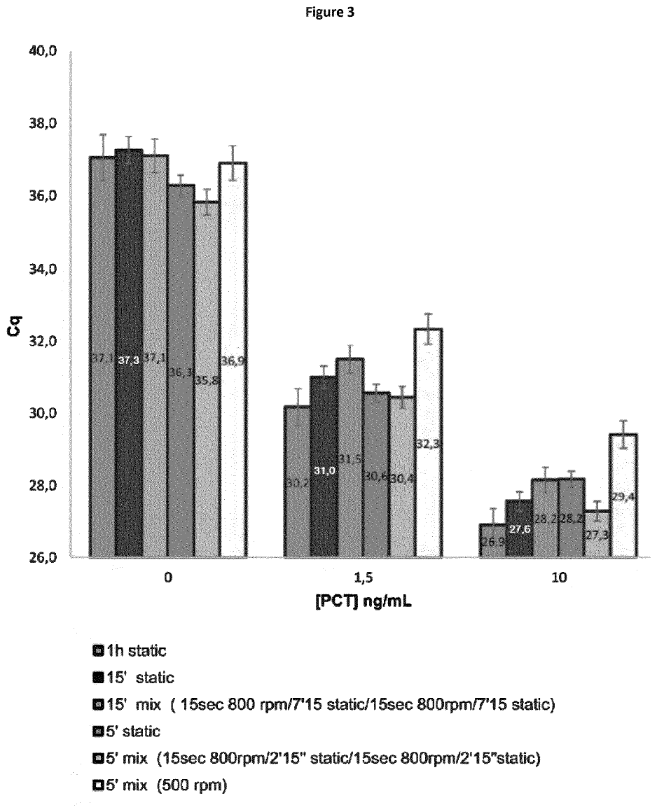

In order to improve the association kinetics over incubation times of 5 or 15 minutes, the binding step was performed with plate shaking using an Eppendorf thermomixer heated to 37° C. This agitation was either continuous (500 rounds per minutes, rpm) or intermittent (agitation at 800 rpm followed by no agitation, repeated twice). The results obtained are summarized in FIG. 3. The reference corresponded to a binding step conducted for 1 h on the same thermomixer but without shaking. The graph shows that 15 min of incubation with intermittent agitation did not bring the Cq at the same level as that observed for the reference. For 5 min of incubation, by adding intermittent agitation, it was possible to decrease the Cq. This effect was more significant for the highest tested concentration of PCT. Interestingly, 5 min of incubation without agitation also led to Cq values equivalent to the reference but only for the lowest tested concentration of PCT. In an unexpected way, continuous agitation at 500 rpm dramatically increased the Cq. Based on Delta-Cq0-10, 1 h of incubation caused the highest difference (Delta-Cq0-10=10.2), closely followed by conditions with 15 min of incubation (8.9 or 9.7, when mixing was added). A 5 min incubation time corresponded to a lower Delta-Cq0-10, with values around 8.3. The lowest Delta-Cq0-10 was obtained with 5 min of incubation with continuous mixing (Delta-Cq0−10=7.5).

TABLE 4

| | | | 15 min | | 5 min | 5 min |
|---|---|---|---|---|---|---|
| | 1 h | 15 min | mix | 5 min | mix | mix |
| Delta-Cq | static | static | & static | static | & static | (500 rpm) |
| 0-10 | 10.17 | 9.7125 | 8.955 | 8.11 | 8.5575 | 7.5 |
| 0-1.5 | 6.895 | 6.27 | 5.615 | 5.73 | 5.4 | 4.5675 |

For Delta-Cq0-1.5 the trend was quite identical, with lowest performance associated to 5 min of incubation with continuous agitation. Interestingly, for Delta-Cq1.5-10 all the values were equivalent (between 2.9 and 3.3), except for 5 min of incubation without mixing, where Delta-Cq1.5-10 was 2.4.

In summary, it was possible to decrease the incubation time down to 15 min without significantly affecting the performance of the assay. For 5 minutes of hybridization, it was better to shake the plate on and off to have Proximity Extension Assay performances approaching those obtained with the reference conditions.

Another step of the Proximity Extension Assay protocol that can potentially be reduced is the 5 min incubation period taking place just after the dilution that follows the binding of the Proximity Extension

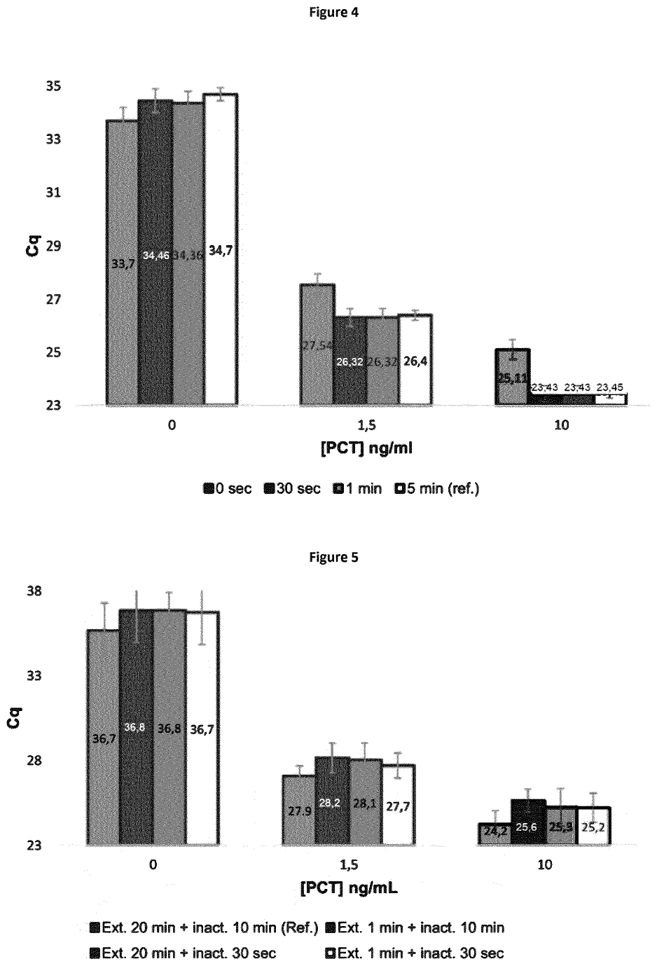

Assay probes to the PCT. As illustrated by the graph presented on FIG. 4, the decrease to 30 sec of the incubation time did not significantly affect the Cq value. However, it was not possible to completely remove this step because a slight but significant reduction of the signal was then observed.

TABLE 5

| Delta Cq | 0 sec | 30 sec | 1 min | 5 min |
|---|---|---|---|---|
| 0-1.5 | 6.16 | 7.9 | 7.7 | 7.9 |
| 0-10 | 10.27 | 11.4 | 10.9 | 10.9 |

The extension step triggered by adding T4 DNA polymerase is crucial because it promotes complementary strand synthesis after hybridization of the two Proximity Extension Assay probes at their 3′-ends. It also promotes background noise reduction by hydrolysing the unmatched Proximity Extension Assay probes thanks to its 3′>5′ exonuclease activity [1]. After an extension step of 20 min at 37° C., the enzyme must be heat-inactivated at 80° C. during 10 min. To assess whether it was possible to reduce the extension time and the inactivation time without impacting the performance of the assay, several experiments were conducted successively. First, the duration of the extension step was reduced from 20 min to 1 minute.

As shown in FIG. 5, no significant difference was observed between the two conditions tested. Second, when the T4 DNA polymerase was inactivated for 30 sec, instead of 10 min at 80° C., using the 20 min-extension protocol, no difference was observed. Finally, when a reduced time of extension step (1 min at 37° C.) was combined with a short time of inactivation, no significant difference with the reference method was observed.

The results, given in Cq, correspond to the mean of three duplicate experiments. The data are also expressed in delta Cq corresponding to the difference between the Cq value obtained for 0 ng/ml of rPCT and the Cq value obtained for different concentrations of rPCT:

TABLE 6

| | 20 min/ | | | |
|---|---|---|---|---|
| | 10 min | 1 min/ | 20 min/ | 1 min/ |
| Delta Cq | (Ref.) | 10 min | 30 sec | 30 sec |
| 0-1.5 | 8.8 | 8.6 | 8.7 | 9 |
| 0-10 | 12.5 | 11.2 | 11.5 | 11.5 |

The whole shortened protocol thus corresponded to 5 min of antigen and Proximity Extension Assay probe binding (with intermittent agitation), followed by 30 sec of incubation after the dilution step and 1 min+30 sec for T4 DNA polymerase extension and inactivation, respectively, for a total incubation time of 7 minutes.

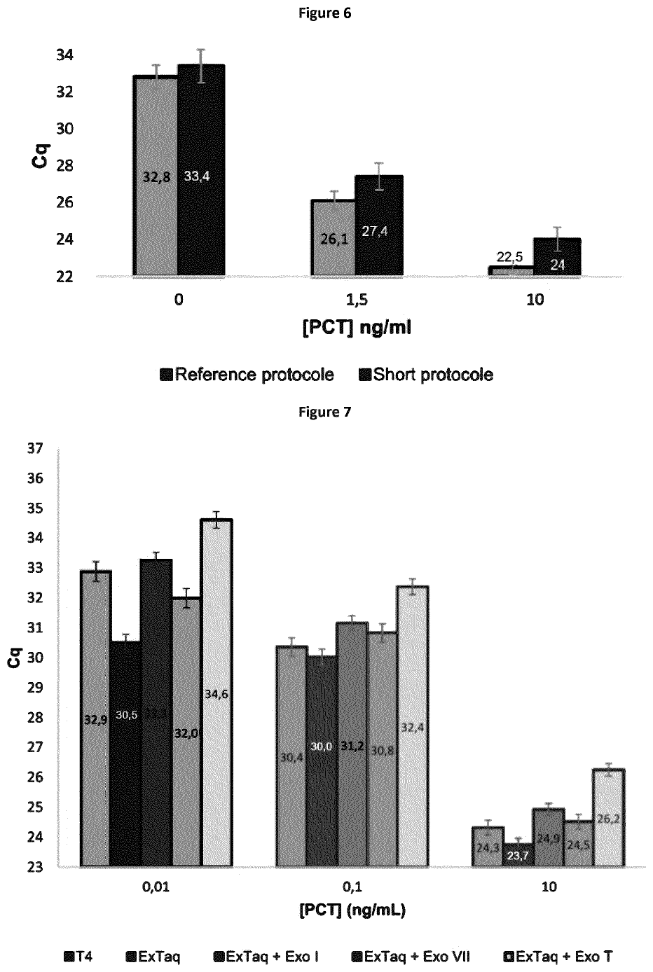

This 7 min protocol was tested in multi-well plates on PBS1x-BSA buffer spiked with rPCT at different concentrations. Results are presented on FIG. 6. Except for 10 ng/ml of rPCT where a small gap of 1.5

Cq was observed, there was no significant difference between the short protocol and the reference protocol for the detection of rPCT.

TABLE 7

| Delta Cq | Reference protocol | Short protocol |
|---|---|---|
| 0-1.5 | 6.7 | 6 |
| 0-10 | 10.3 | 9.34 |

Thus, it is possible to drastically reduce the time needed for Proximity Extension Assay without significantly affecting the performance of the assay for PCT detection.

### Alternative Enzymes to T4 DNA Polymerase

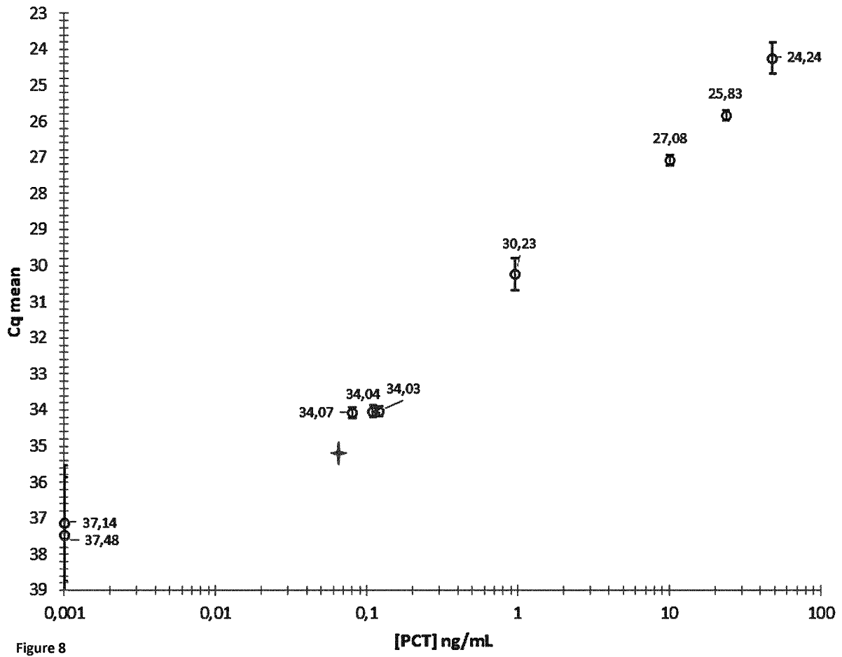

It has been previously demonstrated that 3′>5′ exonuclease activity exhibited by the T4 DNA polymerase reduced nonspecific background and increased Proximity Extension Assay sensitivity [24]. To assess whether it is possible to replace T4 DNA polymerase by another DNA polymerase combining comparable exonuclease activity and higher processivity, a thermostable DNA polymerase having these characteristics was tested (Extaq, Takara). The challenge consisted in eliminating the extension step, by simply adding Extaq to the extension buffer, mixing and then immediately proceeding to the PCR amplification step. As shown in FIG. 7, the use of Extaq instead of T4 resulted in increased background and decreased sensitivity of Proximity Extension Assay. Indeed, the Delta-Cq0-0.1 was 0.5 for the Extaq and 2.5 for the T4 DNA polymerase.

To test the hypothesis that these unsatisfactory results were due to a lower than expected exonuclease activity of Extaq, different exogenous exonucleases (Exonuclease I, Exonuclease VII and Exonuclease T) were evaluated in combination with Extaq. All of these exonucleases, with the exception of Exonuclease T, used in combination with Extaq resulted in restored sensitivity and improved signal-to-noise levels. Exonuclease I and Exonuclease T showed significant improvement in background. However, in terms of Delta-Cq, the performance was similar to that obtained with T4 DNA polymerase: for example, Delta-Cqo-10 was 8.6 for T4 DNA polymerase alone and 8.3 and 8.4 for Extaq combined with Exonuclease I and Exonuclease T, respectively.

TABLE 8

| | | | ExTaq + | ExTaq + | ExTaq + |
|---|---|---|---|---|---|
| Delta Cq | T4 | ExTaq | Exo I | Exo VII | Exo T |
| 0-0.1 | 2.5 | 0.5 | 2.1 | 1.2 | 2.2 |
| 0-10 | 8.6 | 6.8 | 8.3 | 7.5 | 8.4 |

In conclusion, the T4 DNA polymerase can advantageously be replaced by a thermostable DNA polymerase, in terms of background noise reduction, provided that this enzyme exhibits a strong 3>5′ exonuclease activity.

### Implementation of the Short Protocol for PCT Detection in Human Samples

It was crucial to confirm that the fast Proximity Extension Assayprotocol presented henceforth could be applied to real patient plasma samples while maintaining adequate performance. To validate the short Proximity Extension Assayprotocol on real human samples (as opposed to synthetic samples prepared by spiking rPCT expressed in E. Coli into PBS-BSA buffer), the protocol was applied to a variety of human plasmas from several patients having a range of naturel PCT concentrations from 0.08 ng/ml to 48 ng/ml. For this experiment, the probe incubation buffer was complemented by 0.25 mg/ml of purified mouse antibody (Meridian), to mitigate any potential Human Anti-Mouse Antibodies (HAMA) interference [25]. Plasma specimens were obtained from patients with PCT concentrations ranging from the limit of sensitivity required to diagnose bacterial infection (i.e., 0.1 ng/ml) to exceptionally high values (>20 ng/ml), outside the concentration ranges generally observed. A few samples with PCT concentrations above 0.5 ng/ml, indicative of a proven, possibly severe infection, were also tested. These samples were mainly taken from men (5 men and 2 women) and the average age was 66+19 . . . . We did not have access to the patients' medical records. Two PCT negative plasma were also included in this experiment.

The results, which correspond to the average of three independent experiments performed in duplicate, are shown in FIG. 8. It was thus possible, using the short Proximity Extension Assayprotocol developed here, to outline a dose-response curve for different concentrations of PCT in plasma samples from human patients. The Limit of Detection (LoD) was estimated to be 0.063 ng/ml (Cq=35.21). The upper limit of the dynamic range was between 1 and 10 ng/ml.

### DISCUSSION

The main objective of the work presented here was to utilize Proximity Extension Assay for the low-plex detection of markers of medical interest in human samples, and more specifically in the context of decentralized testing, where ease of implementation and short time-to-results are key. More precisely, the aim was to establish a proof of principle for a modified Proximity Extension Assay protocol, more compatible with the short assay times that are expected in the context of point-of-care (POC) clinical diagnostics, generally less than 30 min. This modified Proximity Extension Assay protocol was thus expected to be simpler and faster than the reference Proximity Extension Assay protocol. The biomarker of interest was PCT, which is used in clinical practice for improved patient management in case of suspected bacterial infection, but any other biomarkers of interest could be detected by the short Proximity Extension Assay protocol of the invention.

It has been shown that Proximity Extension Assay performance depends on the affinity of the antibody pair for their respective targets and also on the design of the oligonucleotides used in the Proximity Extension Assay probes. Previous study conducted by Lunberg et al. [8] showed that Proximity Extension Assay probes with a 9 nt hybridization zone gave the highest signal-to-noise level among a variety of probes with hybridization zone of different lengths. The conclusion of the assay developed here was identical: the size of the hybridization zone was found to have a major importance on Proximity Extension Assay sensitivity, with the ideal size being of 9-10 nt long.

It has been demonstrated that it is possible to drastically reduce, by more than 13.5 times, from 95 min to 7 min, the Proximity Extension Assay without significantly affecting the performance of the assay for PCT detection and quantification. The time dedicated to PCT/Proximity Extension Assay probes binding was significantly reduced (from 60 to 5 min). The selection of monoclonal antibody pairs showing high affinity to PCT partially explain these data. It will be necessary, when it is relevant to co-detect one or two additional biomarkers, optionally concomitantly to the PCT, to select antibody pairs with high affinity for the additional target(s).

It has also been demonstrated that intermittent shaking was preferable over continuous agitation. According to inventor's knowledge, it is supposedly because the shearing forces caused by constant shaking did not promote the interaction of ligands to their target. The strength of Proximity Extension Assay is that it combines antibody-based assay with PCR-based amplification of the signal which provides remarkable level of sensitivity and specificity. Signal lost during the shortened time of hybridization may probably be compensated by a gain in signal amplification by PCR.

The incubation times dedicated to the elongation by T4 DNA polymerase were also reduced because if we refer to its polymerase rate of 250-400 nucleotides/see, the complementary stand that it has synthesized was up to 21 nucleotides. Even if the enzyme is considered to have low processivity in the absence of phage T4 accessory proteins, 30 sec seems to be sufficient for complete complementary strand synthesis and background noise reduction [26].

The 30 seconds of denaturation at 80° C. applied after the T4 elongation step appeared to be sufficient to neutralize the enzyme activity, since the results obtained were comparable to those obtained with 10 minutes of denaturation at 80° C. A complementary study not presented here showed that T4 DNA polymerase is very sensitive to heat and can be rapidly inhibited after a few tens of seconds at temperatures between 7° and 80° C.

To reduce the number of steps needed and consequently the time required to perform the complete Proximity Extension Assay protocol, alternative enzymes such as thermostable polymerases with 3′>5′ activity were evaluated. The objective was to perform the Proximity Extension Assay extension and PCR amplification steps using the same enzyme. The hypothesis was that the residual activity of Extaq at 37° C. was sufficient to synthesize the 19 and 21 complementary nucleotides of the duplex. Unfortunately, the performance obtained using Extaq was lower than expected. However, the addition of exogenous exonuclease led to Proximity Extension Assay performance close to that obtained with T4 DNA polymerase, with an additional reduction in background noise. This is further evidence of the crucial role of the 3′>5′ exonuclease activity of the elongation enzyme on the performance and sensitivity of Proximity Extension Assay.

A large number of fully automated immunoassays have become commercially available over the past decade, using different assay formats including enzymatic, luminescent, fluorescent and turbidimetric methods. As the clinical management of patients with severe infections or sepsis is always critical and time-dependent, the availability of fully automated PCT immunoassays with high throughput, short time-to-result, low sample volume, and reasonable cost, is essential [27].

Results of a multi-center evaluation conducted by Dipalo et al. on several commercial systems suggested that the different solutions tested exhibited comparable performance for the diagnostic thresholds of 2.0 and 10 ng/ml of PCT, which reflect moderate and high risk of progression to severe infection and sepsis. The functional sensitivity was generally between 0.01 and 0.05 ng/ml [29].

Nevertheless, Lippi et al. showed considerably lower LoB (limit of blank), LoD (limit of detection) and functional sensitivity values (all≤0.003 ng/mL) for recently commercialized system, better than other fully automated techniques.

For VIDAS® B.R.A.H.M.S PCT™ kit, the analytical limit of detection was estimated to be 0.05 ng/ml and the functional limit of detection was 0.09 ng/ml (https://www.biomerieux-diagnostics.com/vidasr brahms-pct).

Overall, the performance of the assay of the invention is competitive with that of the majority of commercial solutions and should allow for adequate quantification of biomarkers such as PCT at the various diagnostic thresholds that reflect the different stages of a bacterial infection, from local to systemic.

### BIBLIOGRAPHIC REFERENCES

[1] Karam J D, Konigsberg W H. DNA polymerase of the T4-related bacteriophages. Prog Nucleic Acid Res Mol Biol. 2000; 64:65-96. Epub 2000 Mar. 4. doi: 10.1016/s0079-6603 (00) 64002-3. PubMed PMID: 10697407.
[2] Sano T, Smith C L, Cantor C R. Immuno-PCR: very sensitive antigen detection by means of specific antibody-DNA conjugates. Science. 1992;258 (5079): 120-2. Epub 1992 Oct. 2. doi: 10.1126/science. 1439758. PubMed PMID: 1439758.
[3] Adler M, Wacker R, Niemeyer C M. A real-time immuno-PCR assay for routine ultrasensitive quantification of proteins. Biochem Biophys Res Commun. 2003; 308 (2): 240-50. Epub 2003 Aug. 7. doi: 10.1016/s0006-291x (03) 01364-0. PubMed PMID: 12901860.
[4] Barletta J M, Edelman D C, Constantine N T. Lowering the detection limits of HIV-1 viral load using real-time immuno-PCR for HIV-1 p24 antigen. Am J Clin Pathol. 2004; 122 (1): 20-7. Epub 2004 Jul. 27. doi: 10.1309/529t-2wdn-eb6x-8vun. PubMed PMID: 15272526
[5] Fredriksson S, Gullberg M, Jarvius J, Olsson C, Pietras K, Gustafsdóttir S M, et al. Protein detection using proximity-dependent DNA ligation assays. Nat Biotechnol. 2002; 20 (5): 473-7. Epub 2002 May 1. doi: 10.1038/nbt0502-473. PubMed PMID: 11981560.
[6] Nordengrahn A, Gustafsdottir S M, Ebert K, Reid S M, King D P, Ferris N P, et al. Evaluation of a novel proximity ligation assay for the sensitive and rapid detection of foot-and-mouth disease virus. Vet Microbiol. 2008; 127 (3-4): 227-36. Epub 2007 Sep. 28. doi: 10.1016/j.vetmic.2007.08.026. PubMed PMID: 17897794.
[7] Fredriksson S, Dixon W, Ji H, Koong A C, Mindrinos M, Davis R W. Multiplexed protein detection by proximity ligation for cancer biomarker validation. Nat Methods. 2007; 4 (4): 327-9. Epub 2007 Mar. 21. doi: 10.1038/nmeth1020. PubMed PMID: 17369836
[8] Lundberg M, Eriksson A, Tran B, Assarsson E, Fredriksson S. Homogeneous antibody-based proximity extension assays provide sensitive and specific detection of low-abundant proteins in human blood. Nucleic Acids Res. 2011; 39 (15): e102. Epub 2011 Jun. 8. doi: 10.1093/nar/gkr424. PubMed PMID: 21646338; PubMed Central PMCID: PMCPMC3159481
[9] Assarsson E, Lundberg M, Holmquist G, Bjorkesten J, Thorsen S B, Ekman D, et al. Homogenous 96-plex PEA immunoassay exhibiting high sensitivity, specificity, and excellent scalability. PLOS One 2014; 9 (4): e95192. Epub 2014 Apr. 24
[10] Conrad et al., Methods Enzymol. (1996), 267 (Combinatorial Chemistry), 336-367
[11] Fitzgerald, Drug Discov. Today (2000) 5:253-258
[12] Hanes et al., Proc. Natl Acad. Sci. USA (1997) 94:4937-42
[13] Roberts, Curr Opin Chem Biol (1999) Jun; 3:268-73
[14] Schaffitzel et al., J Immunol Methods (1999) December 10; 231:1 19-35
[15] Yin H, Wu Z, Shi N, Qi Y, Jian X, Zhou L, et al. Ultrafast multiplexed detection of SARS-COV-2 RNA using a rapid droplet digital PCR system. Biosens Bioelectron. 2021; 188:113282. Epub 2021 May 22
[16] Schuetz P, Albrich W, Mueller B. Procalcitonin for diagnosis of infection and guide to antibiotic decisions: past, present and future. BMC Med. 2011; 9:107. Epub 2011 Sep. 23.
[17] Wacker C, Prkno A, Brunkhorst F M, Schlattmann P. Procalcitonin as a diagnostic marker forsepsis: a systematic review and meta-analysis. Lancet Infect Dis. 2013; 13 (5): 426-35. Epub 2013 Feb. 5.
[18] Hamade B, Huang D T. Procalcitonin: Where Are We Now? Crit Care Clin. 2020; 36 (1): 23-40. Epub 2019 Nov. 18.
[19] Harbarth S, Holeckova K, Froidevaux C, Pittet D, Ricou B, Grau G E, et al. Diagnostic value of procalcitonin, interleukin-6, and interleukin-8 in critically ill patients admitted with suspected sepsis. AmJ Respir Crit Care Med. 2001; 164 (3): 396-402.
[20] Schendel P F. Overview of protein expression in E. coli. Curr Protoc Mol Biol. 2001;Chapter 16: Unit16.1. Epub 2008 Feb. 12.
[21] Wang G et al. Meth J Immunol 2020
[22] Yokoyama W M. Monoclonal antibody supernatant and ascites fluid production. Curr Protoc Immunol. 2001;Chapter 2: Unit 2.6. Epub 2008 Apr. 25.
[23] Mace D C, Alberts B M. T4 DNA polymerase. Rates and processivity on single-stranded DNA templates. J Mol Biol. 1984; 177 (2): 295-311.
[24] Thorsen S B, Lundberg M, Villablanca A, Christensen S L, Belling K C, Nielsen B S, et al. Detection of serological biomarkers by proximity extension assay for detection of colorectal neoplasias in symptomatic individuals. J Transl Med. 2013; 11:253.
[25] Kricka L J. Human anti-animal antibody interferences in immunological assays. Clin Chem. 1999;45 (7): 942-56. Epub 1999 Jul. 1. PubMed PMID: 10388468.
[26] Spicer E K, Nossal N G, Williams K R. Bacteriophage T4 gene 44 DNA polymerase accessory protein. Sequences of gene 44 and its protein product. J Biol Chem. 1984;259 (24): 15425-32.
[27] Lippi G, Salvagno G L, Gelati M, Pucci M, Demonte D, Faggian D, et al. Analytical Evaluation of the New Beckman Coulter Access Procalcitonin (PCT) Chemiluminescent Immunoassay. Diagnostics (Basel). 2020; 10 (3).
[28] Dipalo M, Guido L, Micca G, Pittalis S, Locatelli M, Motta A, et al. Multicenter comparison of automated procalcitonin immunoassays. Pract Lab Med. 2015; 2:22-8.
[29] Wang D, Caddell B, Nolte F S, Babic N. Comparison of the Abbott Architect BRAHMS and the Biomérieux Vidas BRAHMS Procalcitonin Assays. J Appl Lab Med. 2019; 3 (4): 580-6.

1. An immuno-molecular assay for detecting a protein analyte or a fragment thereof in a biological sample, the assay comprising the steps of:
A) contacting the biological sample with at least one molecule comprising a nucleic acid domain and a binding domain being able to directly or indirectly bind to the protein analyte,
B) incubating the resulting sample from 1 to 30 minutes under intermittent shaking for allowing the at least one molecule to bind to the protein analyte,
C) detecting the binding by a molecular detection method.

2. The method of claim 1, wherein in step B), the sample is incubated from about 3 minutes to about 25 minutes under intermittent shaking.

3. The method of claim 1, wherein the intermittent shaking of step B) comprises alternating periods of shaking lasting from 5 seconds to 30 seconds and static periods lasting from 2 to 3 minutes.

4. The method of claim 1, wherein in step A) the at least one molecule is at least one set of a first and a second proximity probes, each proximity probe comprising an analyte-binding domain and a nucleic acid domain, the analyte-binding domains binding directly to the same protein analyte, and the nucleic acid domains being able to hybridize.

5. The method of claim 4, wherein it comprises the steps of:
I. contacting the biological sample with at least one set of a first and a second proximity probes, each proximity probe comprising an analyte-binding domain and a nucleic acid domain, the analyte-binding domains binding to the same analyte, and the nucleic acid domains being able to hybridize,
II. incubating the resulting sample from 3 to 30 minutes under intermittent shaking, for allowing the proximity probes to bind to the protein analyte and the nucleic acid domains to hybridize with each other to form a duplex structure,
III. diluting the resulting mixture sample with a buffer and incubating further the diluted sample,
IV. adding a DNA polymerase to the sample, and allowing the extension of the 3′ end of at least one nucleic acid domain of the duplex to generate an extension product,
V. heat inactivating the DNA polymerase,
VI. detecting the extension product.

6. The method of claim 5, wherein the polymerase added in step IV) is chosen in the group consisting of: T4 DNA polymerase, T7 DNA polymerase, Extaq polymerase combined with another enzyme having 3′>5′ exonuclease activity, Phi29 (29) DNA polymerase, DNA polymerase I, Klenow fragment of DNA polymerase I, Pyrococcus furiosus (Pfu) DNA polymerase and Pyrococcus woesei (Pwo) DNA polymerase.

7. The method of claim 5, wherein the inactivation step V) is achieved at a temperature comprised between 65° C.-80° C., from 10 seconds to 3 minutes.

8. The method of claim 5, wherein in step II) the sample is incubated from about 3 min to 5 min under intermittent shaking.

9. The method of claim 5, wherein the detecting step VI) is performed by PCR.

10. The method of claim 4, comprising multiplex analysis using several sets of at least first and second proximity probes, wherein each set is specific of a particular protein analyte and produces a unique extension product.

11. The method of claim 1, wherein in step A) the at least one molecule is at least one set of two proximity probes, each proximity probe comprising an analyte-binding domain and a nucleic acid domain, the analyte-binding domains binding directly to the same protein analyte, and the nucleic domains being able to be ligated together.

12. The method of claim 1, wherein in step A) the at least one molecule is a first set of at least two proximity probes each comprising an analyte-binding domain and a second set of at least two proximity probes each comprising a binding domain able to bind to at least one probe of the first set of proximity probes and a nucleic acid domain, the nucleic domains being able to be ligated together.

13. The method of claim 11, wherein it comprises the steps of:
i) contacting the biological sample with at least one set of two proximity probes, each proximity probe comprising an analyte-binding domain and a nucleic acid domain, the analyte-binding domains binding directly to the same protein analyte, and the nucleic domains being able to be ligated together, or contacting the biological sample with a first set of at least two proximity probes each comprising an analyte-binding domain and with a second set of at least two proximity probes each comprising a binding domain able to bind to at least one probe of the first set of proximity probes and a nucleic acid domain, the nucleic domains being able to be ligated together,
j) incubating the resulting sample from 3 to 30 minutes under intermittent shaking for allowing the at least one molecule to bind to the protein analyte,
k) ligating the nucleic acid domains of the proximity probes by a ligase enzyme,
l) detecting the ligated nucleic acids.

14. The method of claim 4, wherein the analyte binding domain of the proximity probes is an antibody, or a binding fragment thereof or a derivative thereof.

15. The method of claim 1, wherein the biological sample is serum or plasma.


SEQUENCE LISTING (2 sequences)

SEQ ID NO: 1 - DNA, 30 bp, synthetic construct
  source 1..30: mol_type = other DNA
taatagtatcgagcggtcagagacgacttc

SEQ ID NO: 2 - DNA, 28 bp, synthetic construct
  source 1..28: mol_type = other DNA
tagctaagtggcaggatgagaagtcgtc